(19) United States
(12) Patent Application Publication
(10) Pub. No.: US 2024/0027643 A1
(43) Pub. Date: Jan. 25, 2024

(54) IDENTIFICATION OF UNIDENTIFIED SUBTERRANEAN SAMPLES

(71) Applicant: Chevron U.S.A. Inc., San Ramon, CA (US)

(72) Inventors: Chao Yan, Sugar Land, TX (US); Wei Wei, Sugar Land, TX (US); Wei Wang, Houston, TX (US)

(21) Appl. No.: 18/358,203

(22) Filed: Jul. 25, 2023

Related U.S. Application Data
(60) Provisional application No. 63/391,823, filed on Jul. 25, 2022.

(51) Int. Cl. G01V 1/50

(52) U.S. Cl. CPC G01V 1/50; G01V 2210/6169

(57) ABSTRACT

A method for identifying an unidentified subterranean sample may include comparing values for a subset of fluid chemistry parameters associated with the unidentified subterranean sample to values of corresponding fluid chemistry parameters associated with a plurality of identified subterranean samples. The method may also include determining, based on comparing the values for the subset of fluid chemistry parameters associated with the unidentified subterranean sample to the values of the corresponding fluid chemistry parameters associated with the plurality of identified subterranean samples, an estimated value for a target parameter associated with the unidentified subterranean sample. The method may further include recategorizing the unidentified subterranean sample as being among the plurality of identified subterranean samples based on the estimated value for the target parameter.

### CROSS-REFERENCE TO RELATED APPLICATIONS

This application claims priority under 35 U.S.C. § 119 to U.S. Provisional Patent Application Ser. No. 63/391,823, titled “Identification of Unidentified Subterranean Samples” and filed on Jul. 25, 2022, the entire contents of which are hereby incorporated herein by reference.

### TECHNICAL FIELD

The present application is related to subterranean field operations and, more particularly, to identifying unidentified subterranean samples.

### BACKGROUND

During various stages of subterranean field operations, subterranean samples are taken and tested for various parameters. At times, a subterranean sample may be identified, for example in terms of the particular wellbore from which the subterranean sample originates, the subterranean formation from which the subterranean sample originates, and/or the depth within a wellbore from which the subterranean sample originates. At other times, however, a subterranean sample may be unidentified, at least to some extent, in terms of its origination. These unidentified subterranean samples may yield important information to help form plans for subsequent stages of a field operation, but only if those subterranean samples may be identified.

### SUMMARY

In general, in one aspect, the disclosure relates to a method for identifying an unidentified subterranean sample. The method may include comparing values for a subset of fluid chemistry parameters associated with the unidentified subterranean sample to values of corresponding fluid chemistry parameters associated with a plurality of identified subterranean samples. The method may also include determining, based on comparing the values for the subset of fluid chemistry parameters associated with the unidentified subterranean sample to the values of the corresponding fluid chemistry parameters associated with the plurality of identified subterranean samples, an estimated value for a target parameter associated with the unidentified subterranean sample. The method may further include recategorizing the unidentified subterranean sample as being among the plurality of identified subterranean samples based on the estimated value for the target parameter.

In another aspect, the disclosure relates to a system for identifying an unidentified subterranean sample. The system may include an analysis apparatus that is configured to compare values for a subset of fluid chemistry parameters associated with the unidentified subterranean sample to values of corresponding fluid chemistry parameters associated with a plurality of identified subterranean samples. The analysis apparatus may also be configured to determine, based on comparing the values for the subset of fluid chemistry parameters associated with the unidentified subterranean sample to the values of the corresponding fluid chemistry parameters associated with the plurality of identified subterranean samples, an estimated value for a target parameter associated with the unidentified subterranean sample. The analysis apparatus may further be configured to recategorize the unidentified subterranean sample as being among the plurality of identified subterranean samples based on the estimated value for the target parameter.

In yet another aspect, the disclosure relates to a computer-implemented method for identifying an unidentified subterranean sample. The method may include obtaining a first plurality of values for a subset of fluid chemistry parameters associated with an unidentified subterranean sample and a second plurality of values of corresponding fluid chemistry parameters associated with a plurality of identified subterranean samples. The method may also include facilitate comparing the first plurality of values to the second plurality of values. The method may further include facilitate determining, based on comparing the first plurality of values to the second plurality of values, an estimated value for a target parameter associated with the unidentified subterranean sample. The method may also include facilitate recategorizing the unidentified subterranean sample as being among the plurality of identified subterranean samples based on the estimated value for the target parameter.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, reference numerals designate like or corresponding, but not necessarily identical, elements.

FIG. 1 shows a field system with which example embodiments may be used.

FIG. 2 shows another field system with which example embodiments may be used.

FIGS. 3A and 3B show detailed views of FIG. 1 according to certain example embodiments.

FIG. 4 shows a diagram of a system for identifying an unidentified subterranean sample according to certain example embodiments.

FIG. 5 shows a system diagram of a controller according to certain example embodiments.

FIG. 6 shows a computing device in accordance with certain example embodiments.

FIG. 7 shows a flowchart of a method for identifying an unidentified subterranean sample according to certain example embodiments.

FIG. 8 shows a hierarchical chart 898 showing how various samples may be correlated according to certain example embodiments.

FIG. 9 shows a graph that correlates the identified subterranean samples of FIG. 8 according to certain example embodiments.

FIG. 10 shows a graph that correlates the unidentified subterranean samples with the identified subterranean samples of FIG. 9 according to certain example embodiments.

FIG. 11 shows a graph that correlates identified subterranean samples according to certain example embodiments.

FIG. 12 shows another graph that correlates identified subterranean samples according to certain example embodiments.

FIGS. 13A through 13C show graphs of production allocation for produced oil for three wells in a well pad according to certain example embodiments.

### DESCRIPTION OF THE INVENTION

The example embodiments discussed herein are directed to systems, apparatus, methods, and devices for identifying unidentified subterranean samples. The subterranean samples identified using example embodiments may include, but are not limited to, oil and natural gas. Identifying unidentified subterranean samples using example embodiments may be designed to comply with certain standards and/or requirements. Example embodiments may be used for wellbores drilled in conventional and/or unconventional (e.g., tight shale) subterranean formations and reservoirs. In some embodiments, an “unconventional” subterranean formation/reservoir may have a permeability of less than 25 millidarcy (mD), such as a permeability of from 0.000001 mD to 25 mD.

Example embodiments may be used to identify unidentified subterranean samples by analyzing measurements of multiple parameters (e.g., fluid chemistry parameters) from the unidentified subterranean samples and from multiple identified subterranean samples. Each of the unidentified subterranean samples has an unknown value for one or more target parameters along with a number of measurable fluid chemistry parameters. Identified subterranean samples originate from a known depth or range of depths from a known wellbore and/or a known formation. Each of the identified subterranean samples has known (e.g., measured) values for the one or more target parameters of each of the unidentified subterranean samples, as well as for multiple fluid chemistry parameters. Example embodiments may provide an estimate for the unknown values of the target parameters for the unidentified subterranean samples. Example embodiments of identifying unidentified subterranean samples may involve subterranean samples that originate from a subsurface (e.g., within and adjacent to a wellbore in a subterranean formation).

Unidentified subterranean samples may be obtained from produced fluid, as a non-limiting example. Based on the knowledge of origin, one could better understand their properties as well as make appropriate decisions, alter operations, etc. However, the traditional geochemistry method may generally only define from which formation these unidentified subterranean samples are generated or produced, but generally cannot provide other critical information such as depth. A typical formation commonly has a vertical distance from 50 feet to hundreds of feet, and only knowing the formation may not be enough information in some instances. Depth of unidentified subterranean samples may help with decision making, altering operations, etc.

As an example, if the estimated depth of an unidentified subterranean sample is determined, more accurate remediation may be performed in the reservoir or production may be avoided from the identified depth. By doing so, hydrocarbon production interruptions may be reduced, especially for deep water wells. As another example, for produced fluid from unconventional fields, due to the complexity of reservoir formation and long horizontal drilling, the fluid source and allocation may be identified to ensure that production is on target formation depth. Comingled fluid from different formation depths could cause incompatibility and lead to loss of production. As yet another example, identification of one or more parameters of an unidentified subterranean sample may be used to optimize spacing between two or more wells and/or the depth of one or more wells.

Embodiments consistent with the instant disclosure may utilize geochemistry parameters which may be correlated to field parameters such as depth, recovery percentage, water cut, etc. Using these correlations, values for field parameters may be estimated and calculated for unidentified subterranean samples. Example embodiments may be used for applications (e.g., biofuels) aside from the production of subterranean resources.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A. In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C. In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of identifying unidentified subterranean samples will be described more fully hereinafter with reference to the accompanying drawings, in which example embodiments of identifying unidentified subterranean samples are shown. Identifying unidentified subterranean samples may, however, be embodied in many different forms and should not be construed as limited to the example embodiments set forth herein. Rather, these example embodiments are provided so that this disclosure will be thorough and complete, and will fully convey the scope of identifying unidentified subterranean samples to those of ordinary skill in the art. Like, but not necessarily the same, elements (also sometimes called components) in the various figures are denoted by like reference numerals for consistency.

Terms such as “first”, “second”, “primary,” “secondary,” “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation, and they are not meant to limit embodiments of identifying unidentified subterranean samples. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

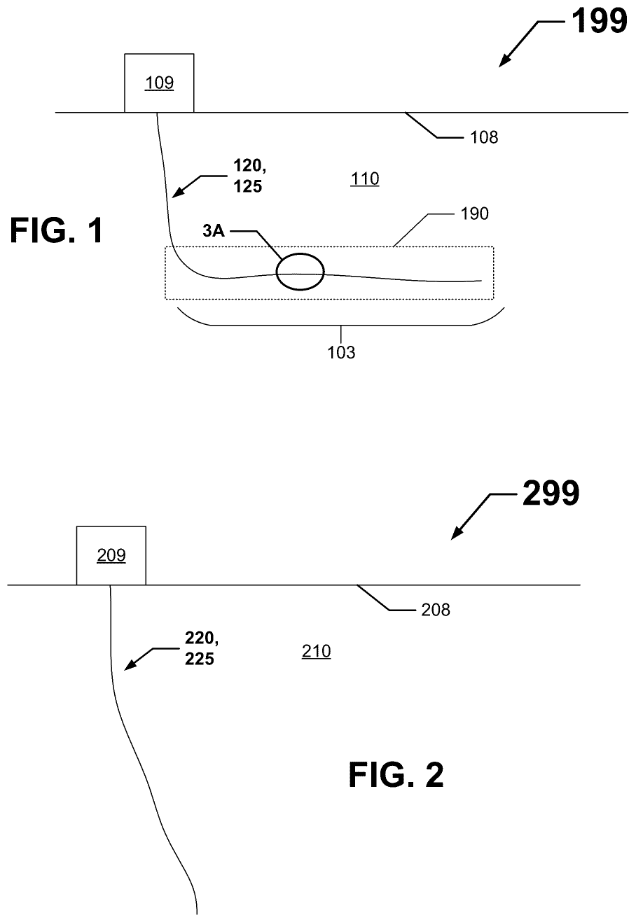

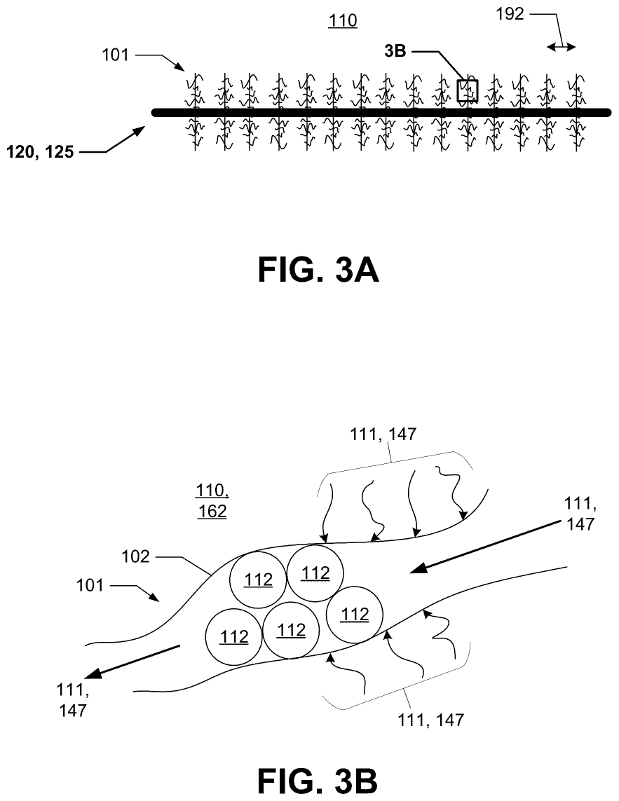

FIG. 1 shows a field system 199 with which example embodiments may be used. Specifically, FIG. 1 shows a schematic diagram of a land-based field system 199 in which a wellbore 120 has been drilled in a subterranean formation 110 and from which subterranean samples may originate. FIG. 2 shows another field system 299 with which example embodiments may be used. Specifically, FIG. 2 shows a schematic diagram of another land-based field system 299 in which a wellbore 220 has been drilled in a subterranean formation 210 and from which subterranean samples may originate. FIG. 3A shows a detail of a substantially horizontal section 103 of the wellbore 120 of FIG. 1. FIG. 3B shows a detail of an induced fracture 101 of FIG. 3A. The field system 199 of FIG. 1 includes a producing wellbore 120 disposed in a subterranean formation 110 using field equipment 109 (e.g., a derrick, a tool pusher, a clamp, a tong, drill pipe, casing pipe, a drill bit, a wireline tool, a fluid pumping system, a subterranean sample testing apparatus) located above a surface 108 and within the wellbore 120. Example embodiments may also apply to other types of wells (e.g., injection wells) that have vertical sections (as in FIGS. 1 and 2) and/or horizontal sections (as in FIG. 1).

With respect to the system 199 of FIG. 1, once the wellbore 120 is drilled, a casing string 125 is inserted into the wellbore 120 to stabilize the wellbore 120 and allow for the extraction of subterranean resources (e.g., natural gas, oil, produced water) from the subterranean formation 110. Field equipment 109, located at the surface 108, is used to drill, encase, fracture, produce, and/or perform any other part of a field operation with respect to the wellbore 120. The wellbore 120 of FIG. 1 starts out as substantially vertical, and then has a substantially horizontal section 103. This configuration of the wellbore 120 is common for exploration and production of subterranean resources, such as oil and natural gas.

Similarly, with respect to the system 299 of FIG. 2, once the wellbore 220 is drilled, a casing string 225 is inserted into the wellbore 220 to stabilize the wellbore 220 from the subterranean formation 210. Field equipment 209, located at the surface 208, is used to drill, encase, fracture, produce, and/or perform any other part of a field operation with respect to the wellbore 220. The wellbore 220 of FIG. 2 is substantially vertical. This configuration of the wellbore 220 is common for injection wells.

Referring back to FIG. 1, the surface 108 may be ground level for an onshore application and the sea floor (or other similar floor under a body of water) for an offshore application. A body of water may include, but it not limited to, sea water, brackish water, flowback or produced water, wastewater (e.g., reclaimed or recycled), brine (e.g., reservoir or synthetic brine), fresh water (e.g., fresh water comprises <1,000 ppm TDS), any other type of water, or any combination thereof. For offshore applications, at least some of the field equipment may be located on a platform that sits above the water level. The point where the wellbore 120 begins at the surface 108 may be called the wellhead. While not shown in FIGS. 1 and 2, there may be multiple wellbores 120, 220, each with its own wellhead but that is located close to the other wellheads, drilled into the subterranean formation 110, 210 and having substantially vertical sections and/or horizontal sections 103 that are close to each other. In such a case, the multiple wellbores 120, 220 may be drilled at the same pad or at different pads.

During the process of drilling the wellbore 120 of FIG. 1 (and similarly for the wellbore 220 of FIG. 2), subterranean samples in the form of cuttings, produced water 147, and/or other subterranean resources 111 (e.g., relatively small amounts of oil or natural gas) are extracted from downhole to the surface 108, where some of the field equipment 109 separates out at least some of the cuttings and recirculates the produced water back downhole. When the drilling process is complete, other operations, such as fracturing operations, may be performed. Throughout each of these various operations, fluids are circulated from the surface 108 by the field equipment 109, and subterranean samples are collected and tested for any of a number of parameters.

While the subterranean formation 110 has naturally-occurring fractures and some fractures that are induced when drilling the wellbore 120, these fractures may need to be enlarged and elongated, and additional fractures need to be induced, in order to extract additional subterranean resources 111 (e.g., oil, natural gas) from the subsurface. In such cases, fracturing operations accomplish these goals. The fractures 101 are shown to be located in the horizontal section 103 of the wellbore 120 in FIG. 3A. The fractures 101, whether induced and/or naturally occurring, may additionally or alternatively be located in other sections (e.g., a substantially vertical section, a transition area between a vertical section and a horizontal section) of a wellbore 120, 220.

The subterranean formation 110 may include one or more of a number of formation types, including but not limited to shale, limestone, sandstone, clay, sand, and salt. In certain embodiments, a subterranean formation 110 may include one or more reservoirs in which one or more resources (e.g., oil, natural gas, water, steam) may be located. One or more of a number of field operations (e.g., fracturing, coring, tripping, drilling, setting casing, extracting downhole resources) may be performed to reach an objective of a user with respect to the subterranean formation 110.

The wellbore 120 may have one or more of a number of segments or hole sections, where each segment or hole section may have one or more of a number of dimensions. Examples of such dimensions may include, but are not limited to, a size (e.g., diameter) of the wellbore 120, a curvature of the wellbore 120, a total vertical depth of the wellbore 120, a measured depth of the wellbore 120, and a horizontal displacement of the wellbore 120. There may be multiple overlapping casing strings of various sizes (e.g., length, outer diameter) contained within and between these segments or hole sections to ensure the integrity of the wellbore construction. In this case, one or more of the segments of the subterranean wellbore 120 is the substantially horizontal section 103.

As discussed above, inserted into and disposed within the wellbore 120 of FIGS. 1 and 3A are a number of casing pipes that are coupled to each other end-to-end to form the casing string 125. Similarly, inserted into and disposed within the wellbore 220 of FIG. 2 are a number of casing pipes that are coupled to each other end-to-end to form the casing string 225. In this case, each end of a casing pipe has mating threads (a type of coupling feature) disposed thereon, allowing a casing pipe to be directly or indirectly mechanically coupled to another casing pipe in an end-to-end configuration. The casing pipes of the casing string 125 and the casing string 225 may be indirectly mechanically coupled to each other using a coupling device, such as a coupling sleeve.

Each casing pipe of the casing string 125 and each casing pipe of the casing string 225 may have a length and a width (e.g., outer diameter). The length of a casing pipe may vary. For example, a common length of a casing pipe is approximately 40 feet. The length of a casing pipe may be longer (e.g., 60 feet) or shorter (e.g., 10 feet) than 40 feet. The width of a casing pipe may also vary and may depend on the cross-sectional shape of the casing pipe. For example, when the shape of the casing pipe is cylindrical, the width may refer to an outer diameter, an inner diameter, or some other form of measurement of the casing pipe. Examples of a width in terms of an outer diameter may include, but are not limited to, 4½ inches, 7 inches, 7⅝ inches, 8⅝ inches, 10¾ inches, 13⅜ inches, and 14 inches.

The size (e.g., width, length) of the casing string 125 and the casing string 225 may be based on the information (e.g., diameter of the borehole drilled) gathered using field equipment with respect to the subterranean wellbore 120 and the subterranean wellbore 220. The walls of the casing string 125 and the casing string 225 have an inner surface that forms a cavity that traverses the length of the casing string 125 and the casing string 225. Each casing pipe may be made of one or more of a number of suitable materials, including but not limited to steel. Cement is poured into the wellbore 120 and the wellbore 220 through the cavity and then forced upward between the outer surface of the casing string 125 and the wall of the subterranean wellbore 120 and between the outer surface of the casing string 225 and the wall of the subterranean wellbore 220. In some cases, a liner may additionally be used with, or alternatively be used in place of, some or all of the casing pipes.

Referring to FIGS. 1, 3A, and 3B, but with principals that may equally apply to FIG. 2, once the cement dries and cures, a number of fractures 101 are induced in the subterranean formation 110. The fractures 101 may be induced in any of a number of ways known in the industry, including but not limited to hydraulic fracturing, fracturing using electrodes, and/or other methods of inducing fractures. The hydraulic fracturing process involves the injection of large quantities of fluids containing water, chemical additives, and proppant 112 into the subterranean formation 110 from the wellbore 120 to create fracture networks. An example of fracturing using electrodes may be found in U.S. Pat. No. 9,840,898 issued on Dec. 12, 2017, to Kasevich et al., the entirety of which is herein incorporated by reference. A subterranean formation 110 naturally has fractures 101, but these naturally occurring fractures 101 have inconsistent characteristics (e.g., length, spacing) and so in some cases cannot be relied upon for extracting subterranean resources without having additional fractures 101, such as what is shown in FIG. 3A, induced in the subterranean formation 110.

Operations that induce fractures 101 in the subterranean formation 110 use any of a number of fluids that include proppant 112 (e.g., sand, ceramic pellets). When proppant 112 is used, some of the fractures 101 (also sometimes called principal or primary fractures) receive proppant 112, while a remainder of the fractures 101 (also sometimes called secondary fractures) do not have any proppant 112 in them.

As shown in FIG. 3B, the proppant 112 is designed to become lodged inside at least some of the induced fractures 101 to keep those fractures 101 open after the fracturing operation is complete. The size of the proppant 112 may be an important design consideration. Sizes (e.g., 40/70 mesh, 50/140 mesh) of the proppant 112 may vary. While the shape of the proppant 112 is shown as being uniformly spherical, and the size is substantially identical among the proppant 112, the actual sizes and shapes of the proppant 112 may vary. If the proppant 112 is too small, the proppant 112 will not be effective at keeping the fractures 101 open enough to effectively allow produced water 147 and/or other subterranean resources 111 to flow through the fractures 101 from the rock matrices 162 in the subterranean formation 110 to the wellbore 120. If the proppant 112 is too large, the proppant 112 may plug up the fractures 101, blocking the flow of the produced water 147 and/or other subterranean resources 111 through the fractures 101.

The use of proppant 112 in certain types of subterranean formations 110, such as shale, is important. Shale formations typically have permeabilities on the order of microdarcys (RD) to nanodarcys (nD). When fractures 101 are induced in such formations with low permeabilities, it is important to sustain the fractures 101 and their permeability and conductivity for an extended period of time in order to extract more of the subterranean resource 111. During fracturing procedures for a production well and during injection for an injection well, the flow of the subterranean resources 111 and the produced water 147 shown in FIG. 3B is replaced in the opposite direction with fracturing fluid and injection fluid, respectively.

The various induced fractures 101 that originate at the wellbore 120 and extend outward into the rock matrices 162 in the subterranean formation 110 in this case have consistent penetration lengths perpendicular to the wellbore 120 and have consistent coverage along at least a portion of the lateral length (substantially horizontal section) of the wellbore 120. For example, induced fractures 101 may be 50 meters high and 200 meters long. Further, the induced fractures 101 may be spaced a distance 192 apart from each other. The distance 192 (e.g., 25 meters, 5 meters, 12 meters) may be optimized based on the permeability and the porosity of the rock matrix 162 of the subterranean formation 110.

The induced fractures 101 create a volume 190 within the subterranean formation 110 where the rock matrix 162 of the subterranean formation 110 is connected to the high conductivity fractures 101 located a short distance away. In addition to different configurations of the fractures 101, other factors that may contribute to the viability of the subterranean formation 110 may include, but are not limited to, permeability of the rock matrix 162, capillary pressure, and the temperature and pressure of the subterranean formation 110. Each fracture 101, whether induced or naturally occurring, is defined by a wall 102, also called a frac face 102 herein. The frac face 102 provides a transition between the paths formed by the rock matrices 162 in the subterranean formation 110 and the fracture 101. The subterranean resources 111 flow through the paths formed by the rock matrices 162 in the subterranean formation 110 into the fracture 101.

The rock matrices 162, as well as the rest of the subterranean formation 110, both without and outside the volume 190, have a certain amount of formation water 147 therein. The produced water 147 may be or include, for example, formation water from the formation matrix within the volume 190, moveable free formation water, and “external” water from non-targeted formation/sources (e.g., outside the target volume 190). These sources of produced water 147 may include water from nearby SWD wells. The produced water 147 may migrate from outside a target volume 190 through other fractures, faults, lineaments, other features of the subterranean formation 110, or any combination thereof.

The produced water 147 may have any of a number of different components (e.g., minerals, chemical additives, acids, completion brine) in addition to formation water. The contents of produced water 147 in one part (e.g., outside the volume 190) of the subterranean formation 110 may be the same as, or different than, the contents of the produced water 147 in other parts (e.g., in the rock matrices 162) of the subterranean formation 110. In some cases, such as during a stage (e.g., a hydraulic fracturing stage) of a field operation, the fluids (e.g., fracturing fluid) used in that stage may mix with or include the produced water 147, thereby changing the contents or composition of the in situ water chemistry in parts (e.g., at or near the fractures 101) of the subterranean formation 110. The produced water 147 may include one or more of a number of types of water, including but not limited to sea water, brackish water, flowback or produced water, wastewater (e.g., reclaimed or recycled), brine (e.g., reservoir or synthetic brine), fresh water (e.g., fresh water comprises <1,000 ppm TDS), any other type of water, or any combination thereof.

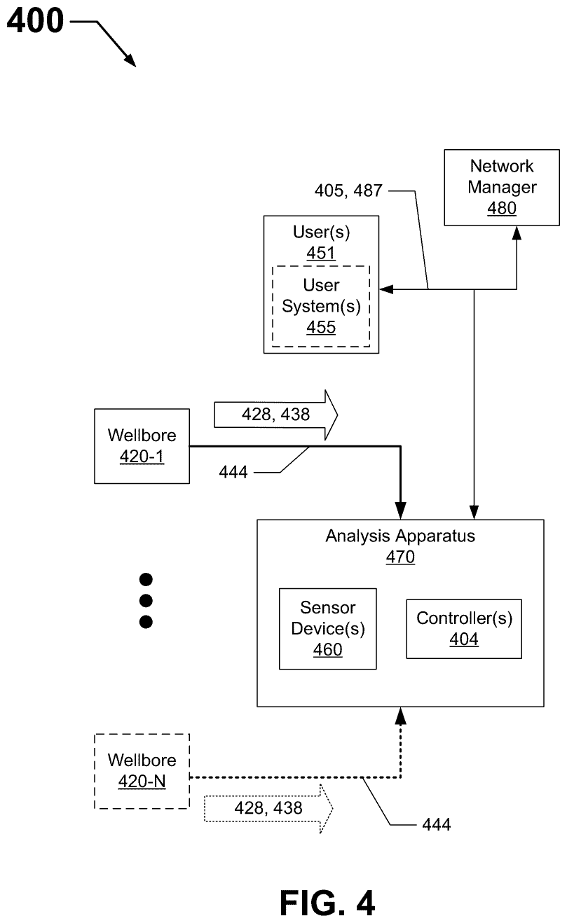

FIG. 4 shows a diagram of a system 400 for identifying an unidentified subterranean sample 438 according to certain example embodiments. The system 400 of FIG. 4 includes one or more wellbores 420, multiple identified subterranean samples 428, one or more unidentified subterranean samples 438, an analysis apparatus 470 (which includes one or more controllers 404 and one or more sensor devices 460), one or more users 451 (including one or more optional user systems 455), a network manager 480, and a conveyance system 444.

The components shown in FIG. 4 are not exhaustive, and in some embodiments, one or more of the components shown in FIG. 4 may not be included in the example testing system 400. Any component of the testing system 400 may be discrete or combined with one or more other components of the testing system 400. Also, one or more components of the testing system 400 may have different configurations. For example, one or more sensor devices 460 may be disposed within or disposed on other components (e.g., the conveyance system 444, a wellbore 420) of the system 400. As another example, one or more controllers 404 may be used to control the conveyance system 444, while one or more other controllers 404 may be used to control and communicate with the sensor devices 460, while one or more other controllers 404 may be used to control the data, run models (forms of algorithms 533, discussed below), and generate estimated values.

Referring to FIGS. 1 through 4, the system 400 may have one or more wellbores 420 (e.g., wellbore 420-1, wellbore 420-N). In this case, there may optionally be N wellbores 420. Each wellbore 420 of the system 400 may be substantially the same as the wellbores discussed above with respect to FIGS. 1 and 2. When there are multiple wellbores 420, two or more wellbores 420 may be from a common pad and/or from different pads. Also, when there are multiple wellbores 420, each wellbore 420 may have characteristics (e.g., total depth, total vertical depth, total length, number of casing stages, size of each casing stage, subterranean formations that are traversed) that may be the same as of different than the characteristics of one or more of the other wellbores 420.

From each wellbore 420, multiple identified subterranean samples 428 are collected. Each identified subterranean sample 428 is taken from a known wellbore 420 (e.g., wellbore 420-1) and at a known depth (e.g., 5600 feet of total vertical depth (TVD)) or range of depths (e.g., between 1200 feet and 1400 feet of wellbore depth). A range of depths of an identified subterranean sample 428 may correspond to a type of geological formation (e.g., shale, sandstone, carbonate) within a subterranean formation (e.g., subterranean formation 110). The wellbore 420 and depth may be parameters of an identified subterranean sample 428. These parameters may be measured or obtained during a field operation at the time that an identified subterranean sample 428 is initially collected or otherwise acquired adjacent to the wellbore 420.

Each identified subterranean sample 428 may also yield values for a number of additional parameters that may be measured using the analysis apparatus 470, as discussed below. At least some of these additional parameters that are measured using the analysis apparatus 470 are fluid chemistry parameters (e.g., oil chemistry parameters). Examples of fluid chemistry parameters may include outputs of analysis performed using equipment such as, but are not limited to, an oil/gas chromatograph, gas chromatograph-mass spectrometry (GC-MS), a stable carbon isotope analysis, a stable sulfur isotope analysis, SARA, a sulfur analysis, a Ni/V analysis, a DNA sequencing analysis, a water analysis, an alkylbenzene analysis, WOGC, a biomarker analysis, and a 2D/3D GC-MS.

Examples of target parameters may include, but are not limited to, temperature, pressure, gas-oil ratio (GOR), oil concentration, mixed oil ratios, oil inflow, original oil in place (OOIP), oil recovery efficiency, hydrogen sulfide content. In some cases, measurements may be made for a large number (e.g., more than 50,000) of parameters from a single identified subterranean sample 428. An identified subterranean sample 428 may be in solid form, liquid form, and/or gaseous form.

Further, one or more unidentified subterranean samples 438 may be collected from one or more of the wellbores 420. Each unidentified subterranean sample 438 is collected without knowing the specific wellbore 420 (e.g., wellbore 420-1) from which the unidentified subterranean sample 438 originates or, alternatively, without knowing the depth or range of depths from a particular wellbore 420 from which the unidentified subterranean sample 438 originates. In addition to these unknown parameters, an unidentified subterranean sample 438 has a number of other parameters whose values may be measured. Example embodiments use the measured values of these parameters of an unidentified subterranean sample 438, in comparison with the measured values of corresponding parameters of multiple identified subterranean samples 428, to estimate the unknown parameters of wellbore 420 and depth within the wellbore 420 so that the unidentified subterranean sample 438 may be recategorized as an identified subterranean sample 428.

Each unidentified subterranean sample 438 may also yield values for a number of additional parameters that may be measured using the analysis apparatus 470, as discussed below. At least some of these additional parameters that are measured using the analysis apparatus 470 are fluid chemistry parameters, which may be substantially the same as the fluid chemistry parameters discussed above with respect to an identified subterranean sample 428. In some cases, measurements may be made for a large number (e.g., more than 50,000) of parameters from a single unidentified subterranean sample 438. An unidentified subterranean sample 438 may be in solid form, liquid form, and/or gaseous form.

Each identified subterranean sample 428 and each unidentified subterranean sample 438 are delivered from the wellbores 420 to the analysis apparatus 470 using the conveyance system 444. The conveyance system 444 may include any equipment and/or modes of transport so that the identified subterranean samples 428 and the unidentified subterranean samples 438 are delivered to the analysis apparatus 470 in a condition that allows multiple parameters associated with the identified subterranean samples 428 and the unidentified subterranean samples 438 to be measured.

The conveyance system 444 may include any equipment that may transport an identified subterranean sample 428 and an unidentified subterranean samples 438, regardless of the state (e.g., solid, liquid, gas, a combination thereof) of the sample. Examples of such equipment may include, but are not limited to, pipes, tubes, valves, storage tanks, pumps, motors, controllers (e.g., controller 404), sensor devices (e.g., sensor device 460), conveyor belts, trucks, rail systems, shipping containers, refer containers, compressors, cranes, test tubes, heaters, coolers, refrigerants, preservatives, and beakers.

The analysis apparatus 470 is configured to perform multiple functions with respect to each of the identified subterranean samples 428 and each of the unidentified subterranean sample 438. For example, the analysis apparatus 470 may be configured to receive each identified subterranean sample 428 and each unidentified subterranean sample 438 delivered by the conveyance system 444. As another example, the analysis apparatus 470 may be configured to measure, using one or more sensor devices 460, one or more parameters associated with each identified subterranean sample 428 and each unidentified subterranean sample 438.

As yet another example, the analysis apparatus 470 may be configured to organize, using one or more of the controllers 404, the measurements of the parameters associated with each identified subterranean sample 428 and each unidentified subterranean sample 438. As still another example, the analysis apparatus 470 may be configured to compare, using one or more of the controllers 404, the measurements of the parameters associated with the identified subterranean samples 428 with the corresponding parameters associated with an unidentified subterranean sample 438.

As yet another example, the analysis apparatus 470 may be configured to estimate, using one or more of the controllers 404, a value of one or more target parameters (e.g., a wellbore 420, a depth or range of depths within the wellbore 420, OOIP, hydrogen sulfide content, percentage of water cut) associated with an unidentified subterranean sample 438. As still another example, the analysis apparatus 470 may be configured to recategorize, using one or more of the controllers 404, an unidentified subterranean sample 438 as an identified subterranean sample 428.

In addition to the one or more controllers 404 and the one or more sensor devices 460, the analysis apparatus 470 may include one or more of a number of other components. Such other components may include, but are not limited to, a testing vessel, a motor, a pump, a compressor, tubing, piping, a valve, a heater, a cooling device, and a compressor. In some cases, the analysis apparatus 470 may be configured to simulate downhole conditions. The analysis apparatus 470 may be configured to process multiple identified subterranean samples 428 and/or multiple unidentified subterranean samples 438 simultaneously. The analysis apparatus 470 may be at a single location or spread over multiple locations. Examples of a location for the analysis apparatus 470 (or portion thereof) may include, but are not limited to, a lab on a land-based rig of above the wellbore 420, a lab on a topsides of a floating platform above the wellbore 420, a lab located remotely from the site of the wellbore 420.

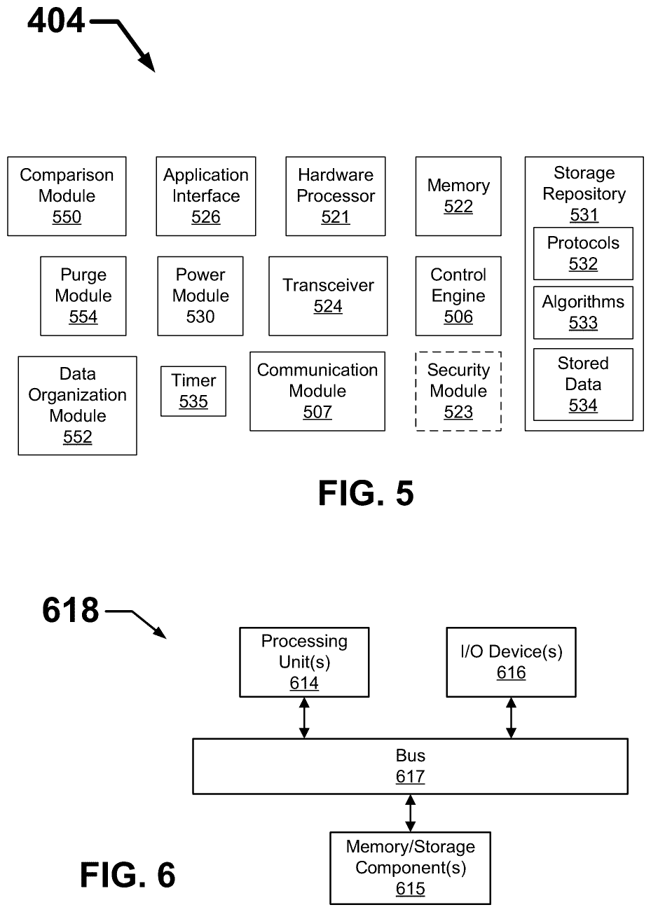

As discussed above, the system 400 may include one or more controllers 404. A controller 404 of the system 400 communicates with and in some cases controls one or more of the other components (e.g., a sensor device 460, part of the conveyance system 444) of the system 400. A controller 404 performs a number of functions that include obtaining and sending data, evaluating data, following protocols, running algorithms, and sending commands. A controller 404 may include one or more of a number of components. As discussed below with respect to FIG. 5, such components of a controller 404 may include, but are not limited to, a control engine, a data organization module, a comparison module, a purge module, a communication module, a timer, a counter, a power module, a storage repository, a hardware processor, memory, a transceiver, an application interface, and a security module. When there are multiple controllers 404 in the system 400, each controller 404 may operate independently of each other. Alternatively, one or more of the controllers 404 may work cooperatively with each other. As yet another alternative, one of the controllers 404 may control some or all of one or more other controllers 404 in the testing system 400. Each controller 404 may be considered a type of computer device, as discussed below with respect to FIG. 6.

Each sensor device 460 includes one or more sensors that measure one or more parameters (e.g., pressure, flow rate, temperature, humidity, mass, weight, fluid content, voltage, current, permeability, porosity, rock characteristics, chemical elements in a fluid, chemical elements in a solid). Examples of a sensor of a sensor device 460 may include, but are not limited to, a temperature sensor, a flow sensor, a pressure sensor, a scale, a mass spectrometer (e.g., a quadrupole mass analyzer, a time of flight mass analyzer, a quadrupole ion trap mass analyzers, an electrostatic sector mass analyzer), a chromatograph, a DNA sequencing apparatus, a sulfur analyzer, a voltmeter, an ammeter, a permeability meter, a porosimeter, and a camera. A sensor device 460 may measure a parameter associated with one or more identified subterranean samples 428 and/or one or more unidentified subterranean samples 438.

A number of different tests may be run using one or more of the sensor devices 460. Such tests may include, but are not limited to, a saturates, aromatics, resins, and asphaltenes (SARA) analysis, a whole oil gas chromatograph analysis, an oil biomarker GC-MS analysis, a stable carbon isotope analysis, a stable sulfur isotope analysis, a nickel/vanadium (Ni/V) analysis, DNA sequencing for oil samples, a water analysis, an alkylbenzene analysis, a 2D/3D GC-MS analysis, a gas-oil ratio (GOR) analysis, an oil concentration analysis, a hydrogen sulfide content analysis, an OOIP analysis, a mixed oil ratio analysis, an oil inflow analysis, WOGC, a biomarker analysis, and an oil recovery efficiency analysis.

A sensor device 460 may also be used with respect to the conveyance system 444. For example, a sensor device 460 may be configured to measure a parameter (e.g., flow rate, pressure, temperature) of a sample (e.g., an identified subterranean sample 428, an unidentified subterranean sample 438) at one or more points in the conveyance system 444. As another example, a sensor device 460 may be configured to determine how open or closed a valve within the conveyance system 444 is. In some cases, a number of sensor devices 460, each measuring a different parameter, may be used in combination to determine and confirm whether a controller 404 should take a particular action (e.g., operate a valve, operate or adjust the operation of the analysis apparatus 470). When a sensor device 460 includes its own controller 404 (or portions thereof), then the sensor device 460 may be considered a type of computer device, as discussed below with respect to FIG. 6.

A user 451 may be any person that interacts, directly or indirectly, with the network manager 480, a controller 404, and/or any other component of the system 400. Examples of a user 451 may include, but are not limited to, a business owner, an engineer, a company representative, a geologist, a consultant, a drilling engineer, a contractor, and a manufacturer's representative. A user 451 may use one or more user systems 455, which may include a display (e.g., a GUI). A user system 455 of a user 451 may interact with (e.g., send data to, obtain data from) the controller 404 via an application interface and using the communication links 405. The user 451 may also interact directly with the network manager 480, a controller 404, and/or some other component of the system 400 through a user interface (e.g., keyboard, mouse, touchscreen).

The network manager 480 is a device or component that controls all or a portion (e.g., a communication network, the controller 404) of the system 400. The network manager 480 may be substantially similar to the controller 404, as described above. For example, the network manager 480 may include a controller that has one or more components and/or similar functionality to some or all of the controller 404. Alternatively, the network manager 480 may include one or more of a number of features in addition to, or altered from, the features of the controller 404. As described herein, control and/or communication with the network manager 480 may include communicating with one or more other components of the same system 400 or another system. In such a case, the network manager 480 may facilitate such control and/or communication. The network manager 480 may be called by other names, including but not limited to a master controller, a network controller, and an enterprise manager. The network manager 480 may be considered a type of computer device, as discussed below with respect to FIG. 6.

Interaction between each controller 404, the sensor devices 460, the users 451 (including any associated user systems 455), the network manager 480, and other components (e.g., the valves, other components of the analysis apparatus 470) of the system 400 may be conducted using communication links 405 and/or power transfer links 487. Each communication link 405 may include wired (e.g., Class 1 electrical cables, Class 2 electrical cables, electrical connectors, Power Line Carrier, RS485) and/or wireless (e.g., Wi-Fi, Zigbee, visible light communication, cellular networking, Bluetooth, Bluetooth Low Energy (BLE), ultrawide band (UWB), WirelessHART, ISA100) technology. A communication link 405 may transmit signals (e.g., communication signals, control signals, data) between each controller 404, the sensor devices 460, the users 451 (including any associated user systems 455), the network manager 480, and the other components of the system 400.

Each power transfer link 487 may include one or more electrical conductors, which may be individual or part of one or more electrical cables. In some cases, as with inductive power, power may be transferred wirelessly using power transfer links 487. A power transfer link 487 may transmit power between each controller 404, the sensor devices 460, the users 451 (including any associated user systems 455), the network manager 480, and the other components of the system 400. Each power transfer link 487 may be sized (e.g., 12 gauge, 18 gauge, 4 gauge) in a manner suitable for the amount (e.g., 480V, 24V, 120V) and type (e.g., alternating current, direct current) of power transferred therethrough.

FIG. 5 shows a system diagram of a controller 404 according to certain example embodiments. Referring to FIGS. 1 through 5, the controller 404 may be substantially the same as a controller 404 discussed above with respect to FIG. 4. The controller 404 includes multiple components. In this case, the controller 404 of FIG. 5 includes a control engine 506, a data organization module 552, a comparison module 550, a purge module 554, a communication module 507, a timer 535, a power module 530, a storage repository 531, a hardware processor 521, a memory 522, a transceiver 524, an application interface 526, and, optionally, a security module 523. The controller 404 (or components thereof) may be located at or near the various components of the system 400. In addition, or in the alternative, the controller 404 (or components thereof) may be located remotely from (e.g., in the cloud, at an office building) the various components of the system 400.

The storage repository 531 may be a persistent storage device (or set of devices) that stores software and data used to assist the controller 404 in communicating with one or more other components of a system, such as the users 451 (including associated user systems 455), the other components of the analysis apparatus 470, the network manager 480, and the sensor devices 460 of the system 400 of FIG. 4 above. In one or more example embodiments, the storage repository 531 stores one or more protocols 532, one or more algorithms 533, and stored data 534.

The protocols 532 of the storage repository 531 may be any procedures (e.g., a series of method steps) and/or other similar operational processes that the control engine 506 of the controller 404 follows based on certain conditions at a point in time. The protocols 532 may include any of a number of communication protocols that are used to send and/or obtain data between the controller 404 and other components of a system (e.g., system 400). Such protocols 532 used for communication may be a time-synchronized protocol. Examples of such time-synchronized protocols may include, but are not limited to, a highway addressable remote transducer (HART) protocol, a wirelessHART protocol, and an International Society of Automation (ISA) 100 protocol. In this way, one or more of the protocols 532 may provide a layer of security to the data transferred within a system (e.g., system 400). Other protocols 532 used for communication may be associated with the use of Wi-Fi, Zigbee, visible light communication (VLC), cellular networking, BLE, UWB, and Bluetooth.

The algorithms 533 may be any formulas, mathematical models, forecasts, simulations, and/or other similar tools that the control engine 506 of the controller 404 uses to reach a computational conclusion. For example, one or more algorithms 533 may be used, in conjunction with one or more protocols 532, to assist the controller 404 to determine when to start, adjust, and/or stop the operation of the analysis apparatus 470 (or portion thereof), the conveyance system 444 (or portion thereof), and/or one or more sensor devices 460.

As another example, one or more algorithms 533 may be used, in conjunction with one or more protocols 532, to allow the analysis apparatus 470 to receive each identified subterranean sample 428 and each unidentified subterranean sample 438 delivered by the conveyance system 444. As another example, one or more algorithms 533 may be used, in conjunction with one or more protocols 532, to allow the analysis apparatus 470 to measure, using one or more sensor devices 460, one or more parameters associated with each identified subterranean sample 428 and each unidentified subterranean sample 438.

As yet another example, one or more algorithms 533 may be used, in conjunction with one or more protocols 532, to allow the analysis apparatus 470 to organize, using one or more of the controllers 404, the measurements of the parameters associated with each identified subterranean sample 428 and each unidentified subterranean sample 438. As still another example, one or more algorithms 533 may be used, in conjunction with one or more protocols 532, to allow the analysis apparatus 470 to compare the measurements of the parameters associated with the identified subterranean samples 428 with the corresponding parameters associated with an unidentified subterranean sample 438.

As yet another example, one or more algorithms 533 may be used, in conjunction with one or more protocols 532, to allow the analysis apparatus 470 to estimate a value of one or more target parameters (e.g., a wellbore 420, a depth or range of depths within the wellbore 420, OOIP, hydrogen sulfide content, percentage of water cut) associated with an unidentified subterranean sample 438. As still another example, one or more algorithms 533 may be used, in conjunction with one or more protocols 532, to allow the analysis apparatus 470 to recategorize an unidentified subterranean sample 438 as an identified subterranean sample 428.

Stored data 534 may be any data associated with a field (e.g., the subterranean formation 110, the fractures 101 adjacent to a wellbore 120), other fields (e.g., other wellbores and subterranean formations), the unidentified subterranean samples 438, the identified subterranean samples 428, the other components (e.g., motors, pumps, compressors) of the system 400, measurements made by the sensor devices 460, threshold values, tables (e.g., for organizing and storing data such as measurements of parameters), results of previously run or calculated algorithms 533, updates to protocols 532 and/or algorithms 533, user preferences, and/or any other suitable data. Such data may be any type of data, including but not limited to historical data, present data, and future data (e.g., forecasts). The stored data 534 may be associated with some measurement of time derived, for example, from the timer 535.

Examples of a storage repository 531 may include, but are not limited to, a database (or a number of databases), a file system, cloud-based storage, a hard drive, flash memory, some other form of solid-state data storage, or any suitable combination thereof. The storage repository 531 may be located on multiple physical machines, each storing all or a portion of the communication protocols 532, the algorithms 533, and/or the stored data 534 according to some example embodiments. Each storage unit or device may be physically located in the same or in a different geographic location.

The storage repository 531 may be operatively connected to the control engine 506. In one or more example embodiments, the control engine 506 includes functionality to communicate with the users 451 (including associated user systems 455), the sensor devices 460, the network manager 480, and/or the other components in the system 400. More specifically, the control engine 506 sends information to and/or obtains information from the storage repository 531 in order to communicate with the users 451 (including associated user systems 455), the sensor devices 460, the network manager 480, and/or the other components of the system 400. As discussed below, the storage repository 531 may also be operatively connected to the communication module 507 in certain example embodiments.

In certain example embodiments, the control engine 506 of the controller 404 controls the operation of one or more components (e.g., the communication module 507, the timer 535, the transceiver 524) of the controller 404. For example, the control engine 506 may activate the communication module 507 when the communication module 507 is in “sleep” mode and when the communication module 507 is needed to send data obtained from another component (e.g., a sensor device 460) in the testing system 400. In addition, the control engine 506 of the controller 404, using one or more algorithms 533 and/or one or more protocols 532, may control (e.g., start, stop, adjust) the operation of one or more other components (e.g., the analysis apparatus 470, the conveyance system 444), or portions thereof, of the system 400.

The control engine 506 of the controller 404, using one or more algorithms 533 and/or one or more protocols 532, may communicate with one or more other components of the system 400. For example, the control engine 506 may use one or more protocols 532 to facilitate communication with the sensor devices 460 to obtain data (e.g., measurements of various parameters, such as temperature, pressure, and flow rate), whether in real time or on a periodic basis and/or to instruct a sensor device 460 to take a measurement. The control engine 506 may use measurements of parameters taken by sensor devices 460, as well as one or more protocols 532 and/or algorithms 533, to determine an estimated value of one or more parameters of an unidentified subterranean sample 438.

The control engine 506 of a controller 404 may also use one or more algorithms 533 and/or one or more protocols 532 to receive each identified subterranean sample 428 and each unidentified subterranean sample 438 delivered by the conveyance system 444. The control engine 506 of a controller 404 may further use one or more algorithms 533 and/or one or more protocols 532 to allow the analysis apparatus 470 to measure, using one or more sensor devices 460, one or more parameters associated with each identified subterranean sample 428 and each unidentified subterranean sample 438.

The control engine 506 of a controller 404 may also use one or more algorithms 533 and/or one or more protocols 532 to allow the analysis apparatus 470 to organize, using one or more of the controllers 404, the measurements of the parameters associated with each identified subterranean sample 428 and each unidentified subterranean sample 438. The control engine 506 of a controller 404 may further use one or more algorithms 533 and/or one or more protocols 532 to allow the analysis apparatus 470 to compare the measurements of the parameters associated with the identified subterranean samples 428 with the corresponding parameters associated with an unidentified subterranean sample 438.

The control engine 506 of a controller 404 may also use one or more algorithms 533 and/or one or more protocols 532 to allow the analysis apparatus 470 to estimate a value of one or more target parameters (e.g., a wellbore 420, a depth or range of depths within the wellbore 420, percentage of water cut, OOIP, hydrogen sulfide content) associated with an unidentified subterranean sample 438. The control engine 506 of a controller 404 may further use one or more algorithms 533 and/or one or more protocols 532 to allow the analysis apparatus 470 to recategorize an unidentified subterranean sample 438 as an identified subterranean sample 428.

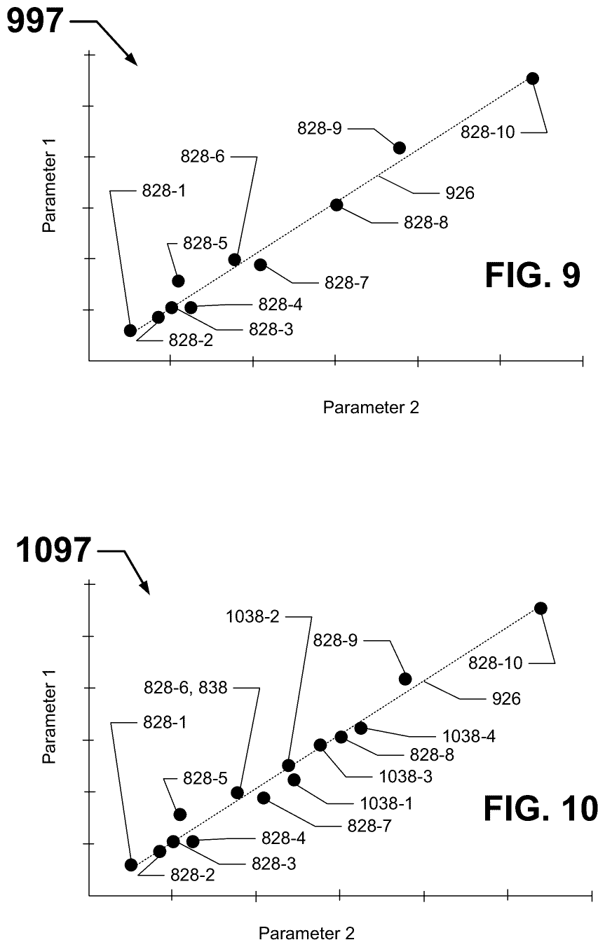

The control engine 506 of a controller 404 may also use one or more algorithms 533 and/or one or more protocols 532 to generate a representation of an unidentified subterranean sample 438, whether before and/or after recategorization. The control engine 506 of a controller 404 may further use one or more algorithms 533 and/or one or more protocols 532 to display or otherwise present, or cause to be displayed or otherwise presented, the representation of an unidentified subterranean sample 438, whether before and/or after recategorization, on a display (or other type of I/O device, such as the I/O device 616 as discussed below with respect to FIG. 6). The control engine 506 of a controller 404 may additionally or alternatively use one or more algorithms 533 and/or one or more protocols 532 to display (e.g., on a user system 455) or otherwise present, or facilitate the display or other type of presentation of, a visual representation (e.g., a graph, a table) of results generated by example embodiments. Examples of such display or other type of presentation are the graphs discussed below with respect to FIGS. 9 through 13C.

The control engine 506 may generate and process data associated with control, communication, and/or other signals sent to and obtained from the users 451 (including associated user systems 455), the sensor devices 460, the network manager 480, the conveyance system 444, and the other components of the system 400. In certain embodiments, the control engine 506 of the controller 404 may communicate with one or more components of a system external to the system 400. For example, the control engine 506 may interact with an inventory management system by ordering replacements for components or pieces of equipment (e.g., a sensor device 460, a valve, a motor) within the system 400 that has failed or is failing. As another example, the control engine 506 may interact with a contractor or workforce scheduling system by arranging for the labor needed to replace a component or piece of equipment in the system 400. In this way and in other ways, the controller 404 is capable of performing a number of functions beyond what could reasonably be considered a routine task.

In certain example embodiments, the control engine 506 may include an interface that enables the control engine 506 to communicate with the sensor devices 460, the user systems 455, the network manager 480, the conveyance system 444, and the other components of the system 400. For example, if a user system 455 operates under IEC Standard 62386, then the user system 455 may have a serial communication interface that will transfer data to the controller 404. Such an interface may operate in conjunction with, or independently of, the protocols 532 used to communicate between the controller 404 and the users 451 (including corresponding user systems 455), the sensor devices 460, the network manager 480, the conveyance system 444, and the other components of the system 400.

The control engine 506 (or other components of the controller 404) may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The communication module 507 of the controller 404 determines and implements the communication protocol (e.g., from the protocols 532 of the storage repository 531) that is used when the control engine 506 communicates with (e.g., sends signals to, obtains signals from) the user systems 455, the sensor devices 460, the network manager 480, the conveyance system 444, and the other components of the system 400. In some cases, the communication module 507 accesses the stored data 534 to determine which communication protocol is used to communicate with another component of the system 400. In addition, the communication module 507 may identify and/or interpret the communication protocol of a communication obtained by the controller 404 so that the control engine 506 may interpret the communication. The communication module 507 may also provide one or more of a number of other services with respect to data sent from and obtained by the controller 404. Such services may include, but are not limited to, data packet routing information and procedures to follow in the event of data interruption.

The timer 535 of the controller 404 may track clock time, intervals of time, an amount of time, and/or any other measure of time. The timer 535 may also count the number of occurrences of an event, whether with or without respect to time. Alternatively, the control engine 506 may perform a counting function. The timer 535 is able to track multiple time measurements and/or count multiple occurrences concurrently. The timer 535 may track time periods based on an instruction obtained from the control engine 506, based on an instruction obtained from a user 451, based on an instruction programmed in the software for the controller 404, based on some other condition (e.g., the occurrence of an event) or from some other component, or from any combination thereof. In certain example embodiments, the timer 535 may provide a time stamp for each packet of data obtained from another component (e.g., a sensor device 460) of the system 400.

The power module 530 of the controller 404 obtains power from a power supply (e.g., AC mains) and manipulates (e.g., transforms, rectifies, inverts) that power to provide the manipulated power to one or more other components (e.g., the timer 535, the control engine 506) of the controller 404, where the manipulated power is of a type (e.g., alternating current, direct current) and level (e.g., 12V, 24V, 120V) that may be used by the other components of the controller 404. In some cases, the power module 530 may also provide power to one or more of the sensor devices 460.

The power module 530 may include one or more of a number of single or multiple discrete components (e.g., transistor, diode, resistor, transformer) and/or a microprocessor. The power module 530 may include a printed circuit board, upon which the microprocessor and/or one or more discrete components are positioned. In addition, or in the alternative, the power module 530 may be a source of power in itself to provide signals to the other components of the controller 404. For example, the power module 530 may be or include an energy storage device (e.g., a battery). As another example, the power module 530 may be or include a localized photovoltaic power system.

The hardware processor 521 of the controller 404 executes software, algorithms (e.g., algorithms 533), and firmware in accordance with one or more example embodiments. Specifically, the hardware processor 521 may execute software on the control engine 506 or any other portion of the controller 404, as well as software used by the users 451 (including associated user systems 455), the network manager 480, the conveyance system 444, and/or other components of the system 400. The hardware processor 521 may be an integrated circuit, a central processing unit, a multi-core processing chip, SoC, a multi-chip module including multiple multi-core processing chips, or other hardware processor in one or more example embodiments. The hardware processor 521 may be known by other names, including but not limited to a computer processor, a microprocessor, and a multi-core processor.

In one or more example embodiments, the hardware processor 521 executes software instructions stored in memory 522. The memory 522 includes one or more cache memories, main memory, and/or any other suitable type of memory. The memory 522 may include volatile and/or non-volatile memory. The memory 522 may be discretely located within the controller 404 relative to the hardware processor 521. In certain configurations, the memory 522 may be integrated with the hardware processor 521.

In certain example embodiments, the controller 404 does not include a hardware processor 521. In such a case, the controller 404 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 404 (or portions thereof) to be programmable and function according to certain logic rules and thresholds without the use of a hardware processor. Alternatively, FPGAs, IGBTs, ICs, and/or similar devices may be used in conjunction with one or more hardware processors 521.

The transceiver 524 of the controller 404 may send and/or obtain control and/or communication signals. Specifically, the transceiver 524 may be used to transfer data between the controller 404 and the users 451 (including associated user systems 455), the sensor devices 460, the network manager 480, the conveyance system 444, and the other components of the system 400. The transceiver 524 may use wired and/or wireless technology. The transceiver 524 may be configured in such a way that the control and/or communication signals sent and/or obtained by the transceiver 524 may be obtained and/or sent by another transceiver that is part of a user system 455, a sensor device 460, the network manager 480, the conveyance system 444, and/or another component of the system 400. The transceiver 524 may send and/or obtain any of a number of signal types, including but not limited to radio frequency signals.

When the transceiver 524 uses wireless technology, any type of wireless technology may be used by the transceiver 524 in sending and obtaining signals. Such wireless technology may include, but is not limited to, Wi-Fi, Zigbee, VLC, cellular networking, BLE, UWB, and Bluetooth. The transceiver 524 may use one or more of any number of suitable communication protocols (e.g., ISA100, HART) when sending and/or obtaining signals.

Optionally, in one or more example embodiments, the security module 523 secures interactions between the controller 404, the users 451 (including associated user systems 455), the sensor devices 460, the network manager 480, the conveyance system 444, and the other components of the system 400. More specifically, the security module 523 authenticates communication from software based on security keys verifying the identity of the source of the communication. For example, user software may be associated with a security key enabling the software of a user system 455 to interact with the controller 404. Further, the security module 523 may restrict receipt of information, requests for information, and/or access to information.

The comparison module 550 of the controller 404 is configured to compare the measurements (e.g., as measured by one or more of the sensor devices 460) of the various parameters associated with an unidentified subterranean sample 438 to the measurements (e.g., as measured by one or more of the sensor devices 460) of the corresponding parameters associated with the identified subterranean samples 428. The comparisons made by the comparison module 550 may be performed using the control engine 506 in combination with one or more protocols 532 and/or one or more algorithms 533. The comparisons made by the comparison module 550 may include tables generated and maintained by the data organization module 552.

In some cases, the comparison module 550 may also determine, using the control engine 506 in combination with one or more protocols 532 and/or one or more algorithms 533, an estimated value for one or more target parameters (e.g., a wellbore 420, a depth or range of depths within the wellbore 420, percentage of water cut, OOIP, hydrogen sulfide content) associated with an unidentified subterranean sample 438. When this task is complete, the comparison module 550 may further recategorize the unidentified subterranean sample 438 as an identified subterranean sample 428. In this way, the measurements for the parameters and the estimated values for the target parameters of the unidentified subterranean sample 438 are added to tables for the corresponding parameters of the identified subterranean sample 428.

The comparison module 550 may use any of a number of techniques, protocols 532, algorithms 533, and/or other methods of comparing the measurements. Such methods may be used to balance a number of goals, such as reducing data, improved accuracy, and performing efficient correlations and pattern finding. Examples of such methods may include, but are not limited to, principal component analysis (PCA), restricted isometry constant (RIC) statistical method, risk management index (RMI) statistical method, artificial intelligence (AI) approaches, and machine learning/deep learning approaches.

The data organization module 552 of the controller 404 is configured to organize the data (e.g., measurements made by the sensor devices 460 of the various parameters associated with the identified subterranean samples 428 and the unidentified subterranean samples 438) stored in the storage repository 531 of the controller 404. The data organization module 552 may organize the data using the control engine 506 in combination with one or more protocols 532 and/or one or more algorithms 533. The data organization module 552 may organize data in tables and/or in any other format. As an example, the data organization module 552 may establish a table for a particular parameter (e.g., water cut, GOR) for identified subterranean samples 428 from a range of depths within a particular wellbore 420.

The purge module 554 of the controller 404 is configured to purge any data that is determined to be unused. Put another way, measurements associated with certain dormant parameters may be purged from the storage repository 531 by the purge module 554. Purges made by the purge module 554 may be executed using the control engine 506 in combination with one or more protocols 532 and/or one or more algorithms 533. For parameters that are purged, the control engine 506 will avoid testing those parameters in future tests (e.g., measurements by sensor devices 460).

A user 451 (including an associated user system 455), the sensor devices 460, the network manager 480, the conveyance system 444, and the other components of the system 400 may interact with the controller 404 using the application interface 526. Specifically, the application interface 526 of the controller 404 obtains data (e.g., information, communications, instructions, updates to firmware) from and sends data (e.g., information, communications, instructions) to the user systems 455 of the users 451, the sensor devices 460, the network manager 480, the conveyance system 444, and/or the other components of the system 400. Examples of an application interface 526 may be or include, but are not limited to, an application programming interface, a web service, a data protocol adapter, some other hardware and/or software, or any suitable combination thereof. Similarly, the user systems 455 of the users 451, the sensor devices 460, the network manager 480, the conveyance system 444, and/or the other components of the system 400 may include an interface (similar to the application interface 526 of the controller 404) to obtain data from and send data to the controller 404 in certain example embodiments.

In addition, as discussed above with respect to a user system 455 of a user 451, one or more of the sensor devices 460, the network manager 480, the conveyance system 444, and/or one or more of the other components of the system 400 may include a user interface. Examples of such a user interface may include, but are not limited to, a graphical user interface, a touchscreen, a keyboard, a monitor, a mouse, some other hardware, or any suitable combination thereof.

The controller 404, the users 451 (including associated user systems 455), the sensor devices 460, the network manager 480, the conveyance system 444, and the other components of the system 400 may use their own system or share a system in certain example embodiments. Such a system may be, or contain a form of, an Internet-based or an intranet-based computer system that is capable of communicating with various software. A computer system includes any type of computing device and/or communication device, including but not limited to the controller 404. Examples of such a system may include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system may correspond to a computer system as described below with regard to FIG. 6.

Further, as discussed above, such a system may have corresponding software (e.g., user system software, sensor device software, controller software). The software may execute on the same or a separate device (e.g., a server, mainframe, desktop personal computer (PC), laptop, PDA, television, cable box, satellite box, kiosk, telephone, mobile phone, or other computing devices) and may be coupled by the communication network (e.g., Internet, Intranet, Extranet, LAN, WAN, or other network communication methods) and/or communication channels, with wire and/or wireless segments according to some example embodiments. The software of one system may be a part of, or operate separately but in conjunction with, the software of another system within the testing system 400.

FIG. 6 illustrates one embodiment of a computing device 618 that implements one or more of the various techniques described herein, and which is representative, in whole or in part, of the elements described herein pursuant to certain example embodiments. For example, a controller 404 (including components thereof, such as a control engine 506, a hardware processor 521, a storage repository 531, a power module 530, and a transceiver 524) may be considered a computing device 618. Computing device 618 is one example of a computing device and is not intended to suggest any limitation as to scope of use or functionality of the computing device and/or its possible architectures. Neither should the computing device 618 be interpreted as having any dependency or requirement relating to any one or combination of components illustrated in the example computing device 618.

The computing device 618 includes one or more processors or processing units 614, one or more memory/storage components 615, one or more input/output (I/O) devices 616, and a bus 617 that allows the various components and devices to communicate with one another. The bus 617 represents one or more of any of several types of bus structures, including a memory bus or memory controller, a peripheral bus, an accelerated graphics port, and a processor or local bus using any of a variety of bus architectures. The bus 617 includes wired and/or wireless buses.

The memory/storage component 615 represents one or more computer storage media. The memory/storage component 615 includes volatile media (such as random access memory (RAM)) and/or nonvolatile media (such as read only memory (ROM), flash memory, optical disks, magnetic disks, and so forth). The memory/storage component 615 includes fixed media (e.g., RAM, ROM, a fixed hard drive, etc.) as well as removable media (e.g., a Flash memory drive, a removable hard drive, an optical disk, and so forth).

One or more I/O devices 616 allow a user 451 to enter commands and information to the computing device 618, and also allow information to be presented to a user (e.g., user 451) and/or other components or devices. Examples of input devices 616 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 618 is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer device 618 includes at least the minimal processing, input, and/or output means necessary to practice one or more embodiments.

Further, those skilled in the art will appreciate that one or more elements of the aforementioned computer device 618 is located at a remote location and connected to the other elements over a network in certain example embodiments. Further, one or more embodiments is implemented on a distributed system having one or more nodes, where each portion of the implementation (e.g., the analysis apparatus 470, the conveyance system 444) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

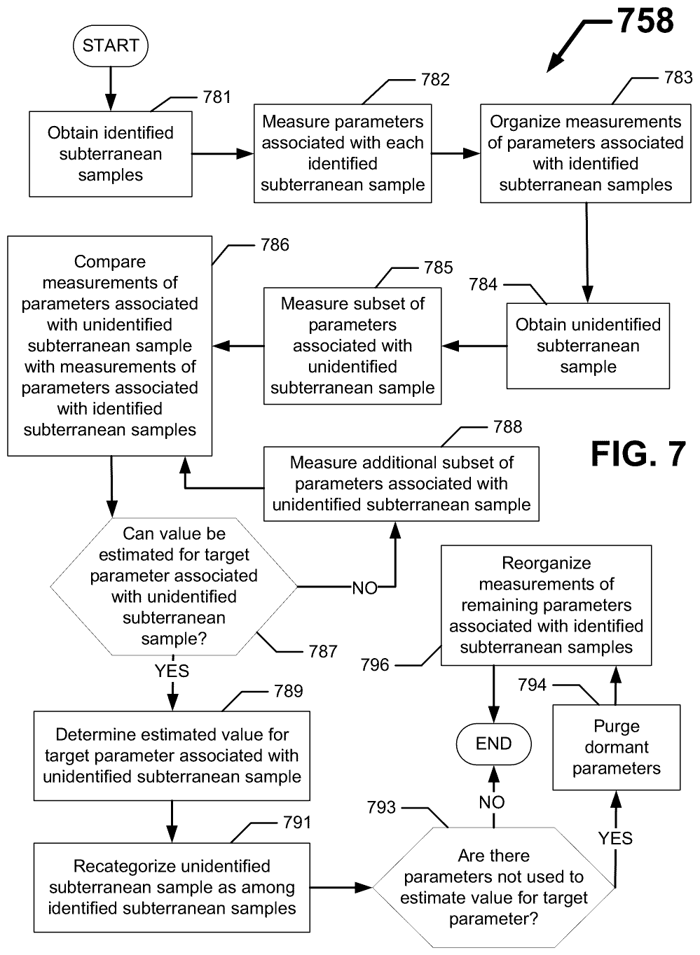

FIG. 7 shows a flowchart 758 of a method for identifying an unidentified subterranean sample 438 according to certain example embodiments. While the various steps in this flowchart 758 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order.

In addition, a person of ordinary skill in the art will appreciate that additional steps not shown in FIG. 7 may be included in performing this method. Accordingly, the specific arrangement of steps should not be construed as limiting the scope. Further, a particular computing device, such as the computing device 618 discussed above with respect to FIG. 6, may be used to perform and/or facilitate one or more of the steps (or portions thereof) for the method shown in FIG. 7 in certain example embodiments. Any of the functions performed and/or facilitated below by a controller 404 may involve the use of one or more protocols 532, one or more algorithms 533, and/or stored data 534 stored in a storage repository 531. In addition, or in the alternative, any of the functions in the method may be performed by a user (e.g., user 451).

The method shown in FIG. 7 is merely an example that may be performed by using an example system described herein. In other words, systems for identifying an unidentified subterranean sample may perform other functions using other methods in addition to and/or aside from those shown in FIG. 7. Referring to FIGS. 1 through 7, the method shown in the flowchart 758 of FIG. 7 begins at the START step and proceeds to step 781, where identified subterranean samples 428 are obtained. As used herein, the term “obtaining” may include receiving, retrieving, accessing, generating, etc. or any other manner of obtaining the identified subterranean samples 428. The identified subterranean samples 428 may be obtained from some or all of the conveyance system 444. The identified subterranean samples 428 may be obtained by the analysis apparatus 470 (or portion thereof).

Some or all of the identified subterranean samples 428 may be obtained using a controller 404 (or an obtaining component thereof), which may include the controller 404 of FIG. above, using one or more algorithms 533 and/or one or more protocols 532. In addition, or in the alternative, some or all of the identified subterranean samples 428 may be obtained from, or with the assistance of, a user 451, including an associated user system 455.

In step 782, one or more parameters associated with the identified subterranean samples 428 are measured. The parameters may be measured by one or more sensor devices 460. At least some of the parameters associated with the identified subterranean samples 428 may be fluid chemistry parameters. The measurements made by some or all of the sensor devices 460 may be at the direction of a controller 404 using one or more algorithms 533 and/or one or more protocols 532. In addition, or in the alternative, the measurements may be performed or directed by a user (e.g., user 451). The measurements may be stored by the controller 404 in the storage repository 531.

Examples of measurements of the parameters associated with the identified subterranean samples 428 may include, but are not limited to, a composition of one or more identified subterranean samples 428, a temperature of one or more identified subterranean samples 428, and a pressure of one or more identified subterranean samples 428. Other examples of measurements of parameters associated with the identified subterranean samples 428 are discussed above with respect to FIGS. 4 and 5. The measurements may be obtained at one time, over a period of time, periodically, or on some other basis. The measurements may be from currently obtained identified subterranean samples 428. In addition, or in the alternative, the measurements may be historical (e.g., measurements obtained from identified subterranean samples 428 of a prior field operation of the subterranean formation 110).

In certain example embodiments, a controller 404 may set and/or control the environment to which the identified subterranean samples 428 are exposed using one or more algorithms 533 and/or one or more protocols 532. For example, if a goal of the testing is to subject the identified subterranean samples 428 to conditions found in the subterranean formation 110, then the controller 404 may accordingly control factors such as the temperature and the pressure applied to the identified subterranean samples 428 when the measurements of the parameters are taken by the sensor devices 460.

In step 783, the measured parameters associated with the identified subterranean samples 428 are organized. The measurements of the parameters may be organized by the data organization module 552 using the control engine 506, one or more algorithms 533, and/or one or more protocols 532. In alternative embodiments, some or all of the measurements may be organized by a user (e.g., user 451). The measurements may be organized in one or more of any of a number of ways. For example, the measurements may be put in specialized tables organized by a range of depths within a wellbore 420. As another example, the measurements may be put in specialized tables organized by particular parameters having values (measurements) within a particular range. The measured parameters, once organized, may be stored in the storage repository 531 and updated as needed.

In step 784, an unidentified subterranean sample 438 is obtained. As used herein, the term “obtaining” may include receiving, retrieving, accessing, generating, etc. or any other manner of obtaining the unidentified subterranean sample 438. The unidentified subterranean samples 438 may be obtained from some or all of the conveyance system 444. The unidentified subterranean sample 438 may be obtained by the analysis apparatus 470 (or portion thereof).

Some or all of the unidentified subterranean sample 438 may be obtained using a controller 404 (or an obtaining component thereof), which may include the controller 404 of FIG. 5 above, using one or more algorithms 533 and/or one or more protocols 532. In addition, or in the alternative, some or all of the unidentified subterranean sample 438 may be obtained from, or with the assistance of, a user 451, including an associated user system 455. In some cases, multiple unidentified subterranean samples 438 are obtained at one time or period of time.

In step 785, one or more parameters associated with the unidentified subterranean sample 438 are measured. The parameters may be measured by one or more sensor devices 460. At least some of the parameters associated with the unidentified subterranean sample 438 may be fluid chemistry parameters. The measurements made by some or all of the sensor devices 460 may be at the direction of a controller 404 using one or more algorithms 533 and/or one or more protocols 532. In addition, or in the alternative, the measurements may be performed or directed by a user (e.g., user 451). The measurements may be stored by the controller 404 in the storage repository 531. The parameters associated with the unidentified subterranean sample 438 that are measured may be the same as, or a subset of, the parameters associated with the identified subterranean sample 428 that are measured in step 782. The measurements may be obtained at one time, over a period of time, periodically, or on some other basis.

In certain example embodiments, a controller 404 may set and/or control the environment to which the unidentified subterranean sample 438 are exposed using one or more algorithms 533 and/or one or more protocols 532. For example, if a goal of the testing is to subject the unidentified subterranean sample 438 to conditions found in the subterranean formation 110, then the controller 404 may accordingly control factors such as the temperature and the pressure applied to the unidentified subterranean sample 438 when the measurements of the parameters are taken by the sensor devices 460.

In step 786, the measurements of the parameters associated with the unidentified subterranean sample 438 is compared with the measurements of the parameters associated with the identified subterranean samples 428. The comparison of the measurements of the parameters associated with the unidentified subterranean sample 438 with the measurements of the parameters associated with the identified subterranean samples 428 may be made using the comparison module 550, the control engine 506, one or more algorithms 533, and/or one or more protocols 532. In some cases, some or all of the comparison of the measurements of the parameters associated with the unidentified subterranean sample 438 with the measurements of the parameters associated with the identified subterranean samples 428 may be performed and/or directed by a user (e.g., user 451).

The comparison of the measurements of some of the parameters may result in a linear correlation. In such a case, selected parameters may be used to perform oil and/or other fluid source rock depth calculations, GOR predictions, water breakthrough predictions, water cut calculations, drain rock volume estimations, reservoir temperature and pressure estimations, oil inflow quantifications, OOIP estimations, oil recovery efficiency monitoring, oil concentration and mixed oil ratio calculations, oil production allocation, asphaltene risk and wax risk quantification, and/or other qualitative findings.

In addition, or in the alternative, the comparison of the measurements of some of the parameters may result in a non-linear correlation. In such a case, non-linear correlations could potentially also be used for a quantitative and/or qualitative determination of variables using approaches including, but not limited to, AI, machine learning/deep learning, and/or other types of modeling approaches. In such cases, developed correlation between oil parameters and variables to quantitatively define variables for unidentified subterranean samples 438 may be used.

If a correlation is non-linear, selected parameters (e.g., some of the fluid chemistry parameters) may be used to perform source rock-oil correlations, perform oil-oil correlations, evaluate reservoir compartmentalization and reservoir connectivity, evaluate well classification, evaluate oil degradation and maturity, evaluate well-well communication, identify oil and/or other fluid sources, perform time-lapse security automation and orchestration (SA&O) monitoring for oil samples, and/or other types of evaluations. In such cases, developed correlation between oil parameters and variables to qualitatively define variables for the unidentified subterranean samples 438 may be used.

In certain example embodiments, the comparison module 550 or some other component (e.g., the control engine 506) of the controller 404 of the analysis apparatus 470, whether for this step 786 and/or for some other step in this method, may be configured to select which algorithm(s) to use among the algorithms 533 stored in the storage repository 531. Similarly, any changes (e.g., based on actual data versus expected results) to an algorithm 533 may be made by the comparison module 550 or some other component of the controller 404.

In step 787, a determination is made as to whether a value may be estimated for a target parameter associated with the unidentified subterranean sample 438. The target parameter may be used to identify the unidentified subterranean sample 438. The determination may be made by the comparison module 550 using the control engine 506, one or more algorithms 533, and/or one or more protocols 532. Alternatively, or in addition, the determination may be made by a user (e.g., user 451). The value for the target parameter may be estimated when enough of the measurements for parameters associated with the unidentified subterranean sample 438 are considered comparable with measurements of the corresponding parameters associated with the identified subterranean samples 428. If a value for the target parameter may be estimated, the process proceeds to step 789. If a value for the target parameter cannot be estimated, the process proceeds to step 788.

In step 788, one or more additional parameters associated with the unidentified subterranean sample 438 are measured. The additional parameters may be measured by one or more sensor devices 460. At least some of the additional parameters associated with the unidentified subterranean sample 438 may be fluid chemistry parameters. The measurements made by some or all of the sensor devices 460 may be at the direction of a controller 404 using one or more algorithms 533 and/or one or more protocols 532. In addition, or in the alternative, the measurements may be performed or directed by a user (e.g., user 451). The measurements may be stored by the controller 404 in the storage repository 531. The additional parameters associated with the unidentified subterranean sample 438 that are measured may be the same as, or a subset of, the parameters associated with the identified subterranean sample 428 that are measured in step 782. The measurements may be obtained at one time, over a period of time, periodically, or on some other basis.

In certain example embodiments, a controller 404 may set and/or control the environment to which the unidentified subterranean sample 438 are exposed using one or more algorithms 533 and/or one or more protocols 532. For example, if a goal of the testing is to subject the unidentified subterranean sample 438 to conditions found in the subterranean formation 110, then the controller 404 may accordingly control factors such as the temperature and the pressure applied to the unidentified subterranean sample 438 when the measurements of the additional parameters are taken by the sensor devices 460. When step 788 is complete, the process may revert to step 786.

In step 789, an estimated value for the target parameter associated with the unidentified subterranean sample 438 is determined. The estimated value for the target parameter may be determined by the comparison module 550 using the control engine 506, one or more algorithms 533, and/or one or more protocols 532. Alternatively, or in addition, the estimated value for the target parameter may be determined by a user (e.g., user 451). The estimated value for the target parameter may be determined based on comparing values for at least some of the fluid chemistry parameters associated with the unidentified subterranean sample 438 to values of corresponding fluid chemistry parameters associated with the identified subterranean samples 428.

For example, if the measured values of some of the fluid chemistry parameters of the unidentified subterranean sample 438 are substantially the same as the values for the same fluid chemistry parameters of a group of the identified subterranean samples 428 that are within the same range of depths (corresponding to the same geographic formation within a subterranean formation (e.g., subterranean formation 110)) of a particular wellbore (e.g., wellbore 420-1). With these correlations, the comparison module 550 and/or a user (e.g., user 451) may determine the estimated value of one or more target parameters (e.g., water cut percentage, GOR, oil concentration, mixed oil ratio, oil inflow, OOIP, oil recovery efficiency, hydrogen sulfide content, range of depths within a wellbore) associated with the unidentified subterranean sample 438 relative to the group of the identified subterranean samples 428.

In step 791, the unidentified subterranean sample 438 is recategorized among the identified subterranean samples 428. The unidentified subterranean sample 438 may be recategorized as part of the group of identified subterranean samples 428 from step 789 by the data organization module 552 using the control engine 506, one or more algorithms 533, and/or one or more protocols 532. In addition, or in the alternative, the unidentified subterranean sample 438 may be recategorized as an identified subterranean sample 428 by a user (e.g., user 451). When the unidentified subterranean sample 438 may be recategorized as an identified subterranean sample 428, the measurements of the parameters of the unidentified subterranean sample 438 are stored and organized with the measurements of the corresponding parameters for the group of identified subterranean samples 428.

In certain example embodiments, the controller 404 (or portions thereof) of the analysis apparatus 470 may implement or facilitate the implementation of one or more field operations (including characteristics (e.g., type, duration, chemicals used, operating parameters, well) thereof). Such field operations may be currently ongoing and/or planned for the future. Such implementation may be based on one or more outputs and/or determinations of this method using example embodiments.

In step 793, a determination is made as to whether some of the parameters associated with the identified subterranean samples 428 are dormant (not used) in terms of estimating a value for a target parameter associated with an unidentified subterranean sample 438. The determination may be made by the purge module 554 using the control engine 506, one or more algorithms 533, and/or one or more protocols 532. Alternatively, or in addition, the determination may be made by a user (e.g., user 451). If some of the parameters associated with the identified subterranean samples 428 are dormant (not used) in terms of estimating a value for a target parameter associated with an unidentified subterranean sample 438, then the process proceeds to step 794. If all of the parameters associated with the identified subterranean samples 428 are used in terms of estimating a value for a target parameter associated with an unidentified subterranean sample 438, then the process proceeds to the END step.

In step 794, the dormant parameters are purged. Measurements for parameters that are not relevant or not used for identifying unidentified subterranean samples 438 occupy valuable resources (e.g., time, digital storage, processing power, sensor devices 460) in the system 400. The dormant parameters may be purged by the purge module 554 using the control engine 506, one or more algorithms 533, and/or one or more protocols 532. Alternatively, or in addition, the dormant parameters may be purged by a user (e.g., user 451).

In step 796, the measurements of the remaining parameters associated with the identified subterranean samples 428 are reorganized. Without the dormant parameters that have been purged, the measurements of the remaining parameters may need to be reorganized. The measurements of the remaining parameters may be reorganized by the data organization module 552 using the control engine 506, one or more algorithms 533, and/or one or more protocols 532. Alternatively, or in addition, the determination may be made by a user (e.g., user 451). When step 796 is complete, the process may proceed to the END step.

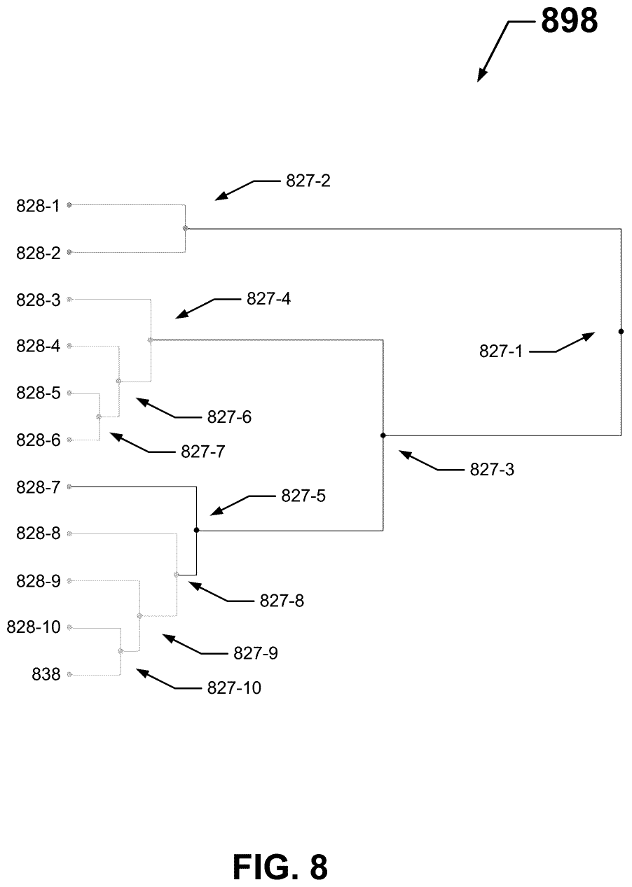

FIG. 8 shows a hierarchical chart 898 showing how various samples may be correlated according to certain example embodiments. Referring to FIGS. 1 through 8, the hierarchical chart 898 includes ten identified subterranean samples 828 and one unidentified subterranean sample 838. The identified subterranean samples 828 and the unidentified subterranean sample 838 in the chart 898 of FIG. 8 are substantially the same as the identified subterranean samples 428 and the unidentified subterranean samples 438 discussed above. Identified subterranean sample 828-1 is identified as originating from wellbore 820-1 at a depth of 280 feet. Identified subterranean sample 828-2 is identified as originating from wellbore 820-2 at a depth of 378 feet. Identified subterranean sample 828-3 is identified as originating from wellbore 820-6 at a depth of 870 feet. Identified subterranean sample 828-4 is identified as originating from wellbore 820-8 at a depth of 1093 feet.

Identified subterranean sample 828-5 is identified as originating from wellbore 820-9 at a depth of 1200 feet. Identified subterranean sample 828-6 is identified as originating from wellbore 820-11 at a depth of 1300 feet. Identified subterranean sample 828-7 is identified as originating from wellbore 820-7 at a depth of 1006 feet. Identified subterranean sample 828-8 is identified as originating from wellbore 820-3 at a depth of 474 feet. Identified subterranean sample 828-9 is identified as originating from wellbore 820-5 at a depth of 778 feet. Identified subterranean sample 828-10 is identified as originating from wellbore 820-4 at a depth of 676 feet. The wellbores 820 of FIG. 8 are substantially the same as the wellbores (e.g., wellbore 420-1) discussed above.

Based on the measurements of one or more parameters (e.g., fluid chemistry parameters) associated with the identified subterranean samples 828 and the unidentified subterranean sample 838, the various samples are correlated and categorized. All of the samples are part of grouping 827-1 based on a correlation (e.g., linear, non-linear) of measurements of some of the parameters of those samples. When considering measurements of additional parameters of those samples, grouping 827-1 is divided into grouping 827-2 (which includes identified subterranean sample 828-1 and identified subterranean sample 828-2) is distinguished from grouping 827-3 (which includes identified subterranean sample 828-3 through identified subterranean sample 828-10 and unidentified subterranean sample 838).

When considering measurements of further additional parameters of those samples, grouping 827-3 is divided into grouping 827-4 (which includes identified subterranean sample 828-3 through identified subterranean sample 828-6) is distinguished from grouping 827-5 (which includes identified subterranean sample 828-7 through identified subterranean sample 828-10 and unidentified subterranean sample 838). When considering measurements of yet further additional parameters of those samples, grouping 827-4 is divided into identified subterranean sample 828-3 and grouping 827-6 (which includes identified subterranean sample 828-4 through identified subterranean sample 828-6). When considering measurements of still further additional parameters of those samples, grouping 827-6 is divided into identified subterranean sample 828-4 and grouping 827-7 (which includes identified subterranean sample 828-5 and identified subterranean sample 828-6).

When considering measurements of yet further additional parameters of those samples, grouping 827-5 is divided into identified subterranean sample 828-7 and grouping 827-8 (which includes identified subterranean sample 828-8 through identified subterranean sample 828-10 and unidentified subterranean sample 838). When considering measurements of still further additional parameters of those samples, grouping 827-8 is divided into identified subterranean sample 828-8 and grouping 827-9 (which includes identified subterranean sample 828-9, identified subterranean sample 828-10, and unidentified subterranean sample 838). When considering measurements of yet further additional parameters of those samples, grouping 827-9 is divided into identified subterranean sample 828-9 and grouping 827-10 (which includes identified subterranean sample 828-10 and unidentified subterranean sample 838).

Through this method, the values of the target parameters (e.g., wellbore 820, depth within the wellbore 820, range of depths within the wellbore 820, percentage of water cut, OOIP, hydrogen sulfide content) of the unidentified subterranean sample 838 may be estimated using example embodiments. In other words, since the measurement of multiple parameters (including multiple fluid chemistry parameters) of the unidentified subterranean sample 838 correlate closely to the measurements of the corresponding parameters of the identified subterranean sample 828-10, the values of the target parameters associated with the unidentified subterranean sample 838 may be estimated.

The estimated values for the target parameters 838 may be precise (e.g., wellbore 820-4, depth of 870 feet) or fall within a range (e.g., wellbore 820-2 through wellbore 820-5, depth of between 800 feet and 950 feet). As a result, the unidentified subterranean sample 838 may be recategorized as an identified subterranean sample 828. The preciseness of the estimated values, whether for an exact value of a range of values, may depend on one or more of a number of factors, including but not limited to the number of identified subterranean samples 828, the relevance of the parameters having measurements, the number of parameters having measurements, and the correlation (e.g., linear, non-linear) between the measurements of different parameters for the identified subterranean samples 828 and/or the unidentified subterranean sample 838.

FIG. 9 shows a graph 997 that correlates the identified subterranean samples of FIG. 8 according to certain example embodiments. Referring to FIGS. 1 through 9, the graph 997 of FIG. 9 plots the ten identified subterranean samples 828 of FIG. 8. The graph 997 is defined by parameter 1 (e.g., GC-MS biomarkers) along the vertical axis and parameter 2 (e.g., GC-MS biomarkers) along the horizontal axis. Parameter 1 and parameter 2 in this case are fluid chemistry parameters that may be measured using one or more types of equipment, including but not limited to an oil/gas chromatograph, a GC-MS, a stable carbon isotope analysis, a stable sulfur isotope analysis, SARA, a sulfur analysis, a Ni/V analysis, a DNA sequencing analysis, a water analysis, an alkylbenzene analysis, WOGC, a biomarker analysis, and a 2D/3D GC-MS. When the 10 identified subterranean samples 828 are plotted on the graph 997 of parameter 1 versus parameter 2, a substantially linear correlation is shown by the line 926 on the graph 997. Statistical analysis, performed by the comparison module 550 of the controller 404 using the control engine 506, one or more protocols 532, and/or one or more algorithms 533, may determine that the linear correlation of the graph 997 has a high statistical significance (e.g., R²=0.9939). The line 926 may have characteristics, as calculated by the comparison module 550, such as a slope (e.g., 1.042) and a Y-intercept value (e.g., 0.05).

FIG. 10 shows a graph 1097 that correlates the unidentified subterranean samples with the identified subterranean samples of FIG. 9 according to certain example embodiments. Referring to FIGS. 1 through 10, the graph 1097 of FIG. 10 includes the ten plots for the identified subterranean samples 828 (identified subterranean sample 828-1, identified subterranean sample 828-2, identified subterranean sample 828-3, identified subterranean sample 828-4, identified subterranean sample 828-5, identified subterranean sample 828-6, identified subterranean sample 828-7, identified subterranean sample 828-8, identified subterranean sample 828-9, and identified subterranean sample 828-10) and the line 926 from the graph 997 of FIG. 9. In addition, the graph 1097 of FIG. 10 includes a plot for the unidentified subterranean sample 838 of FIG. 8.

Further, the graph 1097 of FIG. 10 includes a plot for four additional unidentified subterranean samples 1038 (unidentified subterranean sample 1038-1, unidentified subterranean sample 1038-2, unidentified subterranean sample 1038-3, and unidentified subterranean sample 1038-4). Using the line 926 from the graph 1097 established by the linear correlation of the identified subterranean samples 828, the depths from which the unidentified subterranean sample 838 and the unidentified subterranean samples 1038 originate may be estimated. As a result, the comparison module 550, using the control engine 506, one or more of the protocols 532, and/or one or more of the algorithms 533, may estimate that the depth from which unidentified subterranean sample 838 originates is 870 feet, that the depth from which unidentified subterranean sample 1038-1 and unidentified subterranean sample 1038-2 originate is 689 feet, that the depth from which unidentified subterranean sample 1038-3 originates is 542 feet, and that the depth from which unidentified subterranean sample 1038-4 originates is 447 feet.

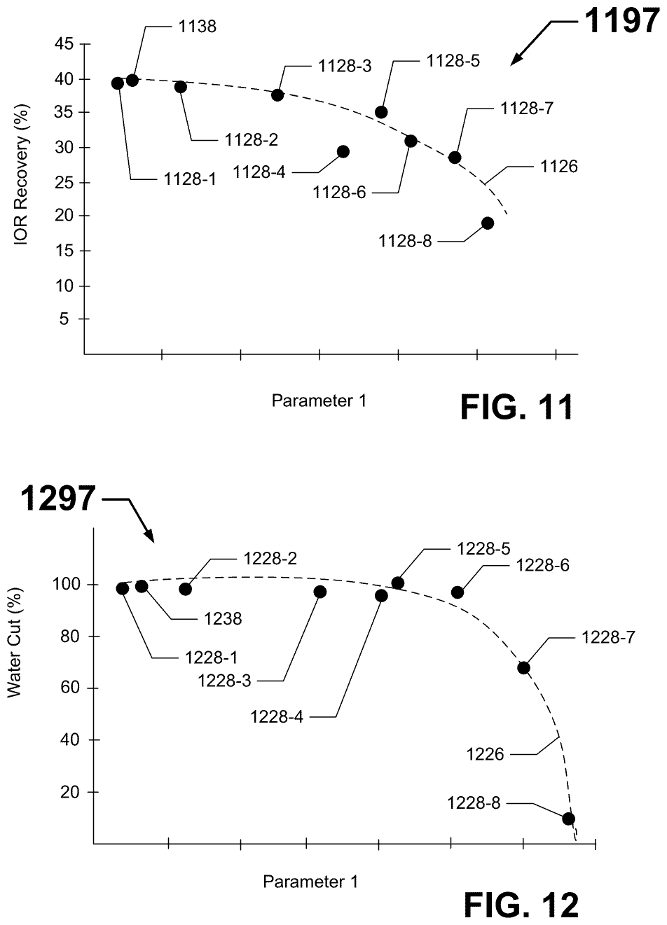

FIG. 11 shows a graph 1197 that correlates identified subterranean samples according to certain example embodiments. Referring to FIGS. 1 through 11, the graph 1197 of FIG. 11 includes 8 plots for identified subterranean samples 1128 (identified subterranean sample 1128-1, identified subterranean sample 1128-2, identified subterranean sample 1128-3, identified subterranean sample 1128-4, identified subterranean sample 1128-5, identified subterranean sample 1128-6, identified subterranean sample 1128-7, and identified subterranean sample 1128-8), one plot for an unidentified subterranean sample 1138, and a curve 1126 that represents the best fit for the data points. The graph 1197 is defined by a fluid chemistry parameter along the horizontal axis and a target parameter in the form of IOR recovery percentage along the vertical axis.

The curve 1126 may be calculated by the comparison module 550 using the control engine 506, one or more of the protocols 532, and/or one or more of the algorithms 533. For example, the curve 1126 may be defined by the equation: y=−1856x²+3742x−973, and with a R² value of 0.7396. The parameter that defines the horizontal axis and the target parameter are both known for the 8 identified subterranean samples 1228, and so the plots for the identified subterranean samples 1228 are based on known data. The value for the parameter that defines the horizontal axis may be measured for the unidentified subterranean sample 1238, and that measurement, combined with the curve 1126, may be used to estimate the value of the target parameter (the IOR recovery percentage) at approximately 40%.

FIG. 12 shows another graph 1297 that correlates identified subterranean samples according to certain example embodiments. Referring to FIGS. 1 through 12, the graph 1297 of FIG. 12 includes 8 plots for identified subterranean samples 1228 (identified subterranean sample 1228-1, identified subterranean sample 1228-2, identified subterranean sample 1228-3, identified subterranean sample 1228-4, identified subterranean sample 1228-5, identified subterranean sample 1228-6, identified subterranean sample 1228-7, and identified subterranean sample 1228-8), one plot for an unidentified subterranean sample 1238, and a curve 1226 that represents the best fit for the data points. The graph 1297 is defined by a fluid chemistry parameter along the horizontal axis and a target parameter in the form of water cut percentage along the vertical axis.

The curve 1226 may be calculated by the comparison module 550 using the control engine 506, one or more of the protocols 532, and/or one or more of the algorithms 533. For example, the curve 1226 may be defined by the equation: y=−3631856x⁵+97543742x⁴−864329841x³+6392840x²−10984732x+49145973, and with a R² value of 1.0. The parameter that defines the horizontal axis and the target parameter are both known for the 8 identified subterranean samples 1228, and so the plots for the identified subterranean samples 1228 are based on known data. The value for the parameter that defines the horizontal axis may be measured for the unidentified subterranean sample 1238, and that measurement, combined with the curve 1226, may be used to estimate the value of the target parameter (the water cut percentage) at approximately 99%.

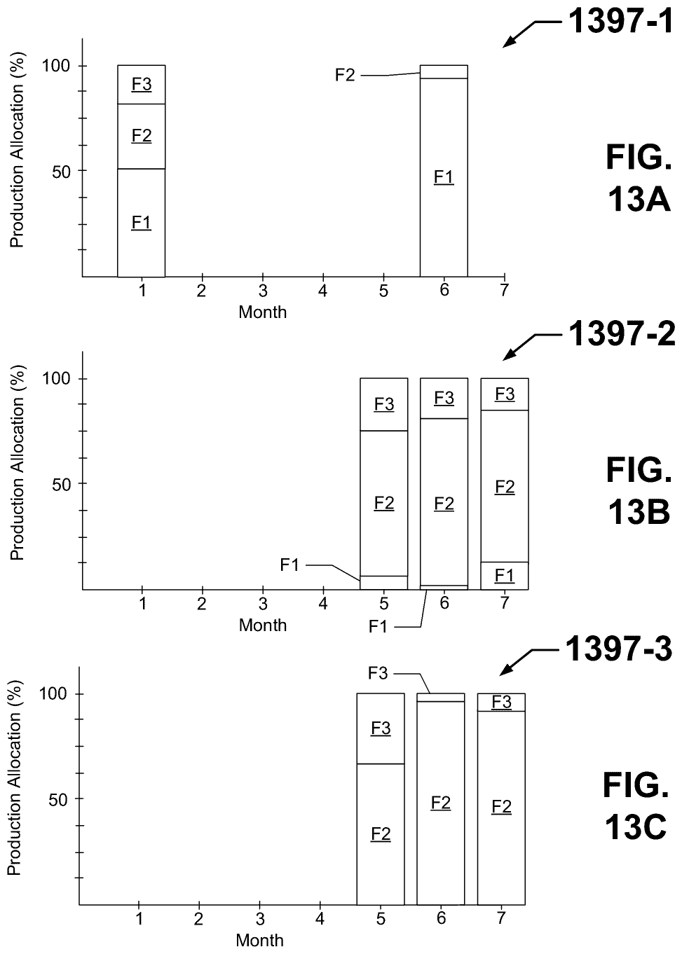

FIGS. 13A through 13C show graphs 1397 of production allocation of produced oil for three wells in a well pad traversing three adjacent subterranean formations according to certain example embodiments. Specifically, graph 1397-1 shows the production allocation of produced oil of a first well (e.g., well 420-1). Graph 1397-2 shows the production allocation of produced oil of a second well (e.g., well 420-2). Graph 1397-3 shows the production allocation of produced oil of a third well (e.g., well 420-3). Referring to the information discussed above with respect to FIGS. 1 through 12, graph 1397-1 shows that the production allocation of produced oil of the first well in month 1 (the first month where production allocation data is collected in this example) is approximately 50% from formation 1 (F1), approximately 35% from formation 2 (F2), and approximately 15% from formation 3 (F3). By contrast, the production allocation of produced oil of the first well in month 7 (five months after month 1) is approximately 95% from F1, approximately 5% from F2, and 0% from F3. The production allocation of produced oil of the first well for months 2 through 5 are not shown in the graph 1397-1.

Graph 1397-2 shows that the production allocation of produced oil of the second well in month 5 (i.e., the second well is new relative to the first well) is approximately 5% from F1, approximately 75% from F2, and approximately 20% from F3. In month 6, the production allocation of produced oil of the second well is approximately 2% from F1, approximately 83% from F2, and approximately 15% from F3. In month 7, the production allocation of produced oil of the second well is approximately 9% from F1, approximately 79% from F2, and approximately 12% from F3. The data shows that the production allocation of produced oil for the second well for months 5-7 is substantially constant.

Graph 1397-3 shows that the production allocation of produced oil of the third well in month 5 (i.e., the third well is new relative to the first well) is 0% from F1, approximately 63% from F2, and approximately 37% from F3. In month 6, the production allocation of produced oil of the third well is 0% from F1, approximately 3% from F2, and approximately 97% from F3. In month 7, the production allocation of produced oil of the third well is 0% from F1, approximately 5% from F2, and approximately 95% from F3.

The graphs 1397 show that the second and third wells, once put on production, may alter the production allocation of the first well without making any substantial changes to the operation of the first well. For example, if F1 is the target formation for the first well, the production allocation of produced oil for the first well in month 1, where F1 is only approximately 50%, may be suboptimal. Using example embodiments, in month 6, after the second and third wells are put on production, the production allocation of produced oil for the first well of approximately 95% from F1 may be considered an optimal result.

Further, if F2 is the target formation for the second well, and if F3 is the target formation for the third well, then the graphs 1397 show that, in addition to contributing to the optimal focus of formation 1 in the production allocation of produced oil for the first well, example embodiments may forecast and/or recognize that the production allocation of produced oil for the second well is optimally and substantially allocated to F2 and that the production allocation of produced oil for the third well is optimally and substantially allocated to F3 by month 6, which is the second month in which the second and third wells are put on production.

In this example, example embodiments may be used to quantify production allocation values for each well and/or for each formation within a well. Specifically, for example, an iteration of values of corresponding fluid chemistry parameters associated with identified subterranean samples and unidentified subterranean samples may be used to quantify production allocation of unidentified subterranean samples from a different formation, whether from the same well or different wells (as in this example).

Example embodiments may be used to identify subterranean samples that have one or more unknown parameters that would otherwise identify the subterranean samples. The unknown parameters may include, but are not limited to, a wellbore, a depth within the wellbore, and a range of depths within the wellbore. Example embodiments may be used to fully or partially automate the process of identifying unidentified subterranean samples. Example embodiments may use a large volume of measurements of a large number of parameters associated with each subterranean sample, whether identified or unidentified. Example embodiments may also analyze the large volume of measurements and purge measurements of parameters that are not used or rarely used. Example embodiments may provide a number of benefits. Such benefits may include, but are not limited to, ease of use, providing further transparency and analysis into particular wellbores to optimize the life of a producing well, flexibility, configurability, self-adjustment and self-correction, and compliance with applicable industry standards and regulations.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the pre sent disclosure will suggest themselves to practitioners of the art. Therefore, the scope of the example embodiments is not limited herein.

What is claimed is:

1. A method for identifying an unidentified subterranean sample, the method comprising:
comparing values for a subset of fluid chemistry parameters associated with the unidentified subterranean sample to values of corresponding fluid chemistry parameters associated with a plurality of identified subterranean samples;
determining, based on comparing the values for the subset of fluid chemistry parameters associated with the unidentified subterranean sample to the values of the corresponding fluid chemistry parameters associated with the plurality of identified subterranean samples, an estimated value for a target parameter associated with the unidentified subterranean sample; and
recategorizing the unidentified subterranean sample as being among the plurality of identified subterranean samples based on the estimated value for the target parameter.

2. The method of claim 1, further comprising:
measuring, using a sensor device, the values for the subset of fluid chemistry parameters associated with the unidentified subterranean sample before comparing the values for the subset of fluid chemistry parameters associated with the unidentified subterranean sample to the values of corresponding fluid chemistry parameters associated with a plurality of identified subterranean samples.

3. The method of claim 1, further comprising:
identifying an unknown value for the target parameter associated with the unidentified subterranean sample before determining the estimated value for the target parameter.

4. The method of claim 1, wherein the target parameter comprises at least one of a group consisting of a depth within a subterranean formation, a produced fluid source, a water cut, original oil in place, hydrogen sulfide content, a recovery percentage of water flood, a gas-oil-ratio, drained rock volume, reservoir temperature, reservoir pressure, wax risk, asphaltene risk, and an allocation.

5. The method of claim 1, wherein the plurality of identified subterranean samples are taken from a common subterranean formation from which the unidentified subterranean sample is taken.

6. The method of claim 5, wherein the plurality of identified subterranean samples are taken from the common wellbore drilled through the subterranean formation, and wherein each of the plurality of identified subterranean samples are taken from a horizontal component or a vertical component of the common wellbore.

7. The method of claim 5, wherein the plurality of identified subterranean samples are taken from multiple wellbores drilled through the subterranean formation, and wherein the multiple wellbores are from a common pad.

8. The method of claim 5, wherein the plurality of identified subterranean samples are taken from a produced fluid collected at the surface.

9. The method of claim 1, wherein the fluid chemistry parameters are measured using at least one of a group consisting of an oil/gas chromatograph, a GC-MS, a stable carbon isotope analysis, a stable sulfur isotope analysis, SARA, a sulfur analysis, a Ni/V analysis, a DNA sequencing analysis, a water analysis, an alkylbenzene analysis, WOGC, a biomarker analysis, and a 2D/3D GC-MS.

10. The method of claim 1, wherein the estimated value for the target parameter associated with the unidentified subterranean sample is quantitively determined using a linear correlation analysis with the values of the corresponding fluid chemistry parameters associated with the plurality of identified subterranean samples.

11. The method of claim 1, wherein the estimated value for the target parameter associated with the unidentified subterranean sample is quantitively determined using a non-linear correlation analysis with the values of the corresponding fluid chemistry parameters associated with the plurality of identified subterranean samples.

12. The method of claim 1, further comprising:
identifying a dormant subset of the corresponding fluid chemistry parameters associated with the plurality of identified subterranean samples that are not among the subset of fluid chemistry parameters associated with of the unidentified subterranean sample; and
removing the dormant subset from among the corresponding fluid chemistry parameters associated with the plurality of identified subterranean samples.

13. The method of claim 1, further comprising:
quantifying a production allocation of an additional unidentified subterranean sample using the values of corresponding fluid chemistry parameters associated with the plurality of identified subterranean samples, wherein the plurality of identified subterranean samples is from a first formation, and wherein the additional unidentified subterranean sample is from a second formation.

14. A system for identifying an unidentified subterranean sample, the system comprising:
an analysis apparatus that is configured to:
compare values for a subset of fluid chemistry parameters associated with the unidentified subterranean sample to values of corresponding fluid chemistry parameters associated with a plurality of identified subterranean samples;
determine, based on comparing the values for the subset of fluid chemistry parameters associated with the unidentified subterranean sample to the values of the corresponding fluid chemistry parameters associated with the plurality of identified subterranean samples, an estimated value for a target parameter associated with the unidentified subterranean sample; and
recategorize the unidentified subterranean sample as being among the plurality of identified subterranean samples based on the estimated value for the target parameter.

15. The system of claim 14, further comprising:
a sensor device configured to measure the values for the subset of fluid chemistry parameters associated with the unidentified subterranean sample before comparing the values for the subset of fluid chemistry parameters associated with the unidentified subterranean sample to the values of the corresponding fluid chemistry parameters associated with a plurality of identified subterranean samples.

16. The system of claim 15, further comprising:
a controller communicably coupled to the sensor device and the analysis apparatus, wherein the controller is configured to control the sensor device and communicate measurements of the values for the subset of fluid chemistry parameters associated with the unidentified subterranean sample to the analysis apparatus.

17. The system of claim 16, further comprising:
a storage repository communicably coupled to the controller, wherein the storage repository is configured to store a plurality of algorithms that correlate the values of the corresponding fluid chemistry parameters associated with the plurality of identified subterranean samples, and wherein the controller is further configured to modify the plurality of algorithms based on future values of the corresponding parameters associated with the plurality of identified subterranean samples.

18. The system of claim 14, wherein the analysis apparatus is further configured to identify an unknown value for the target parameter associated with the unidentified subterranean sample before determining the estimated value for the target parameter.

19. A computer-implemented method for identifying an unidentified subterranean sample, the method comprising:
obtaining a first plurality of values for a subset of fluid chemistry parameters associated with an unidentified subterranean sample and a second plurality of values of corresponding fluid chemistry parameters associated with a plurality of identified subterranean samples;
facilitate comparing the first plurality of values to the second plurality of values;
facilitate determining, based on comparing the first plurality of values to the second plurality of values, an estimated value for a target parameter associated with the unidentified subterranean sample; and
facilitate recategorizing the unidentified subterranean sample as being among the plurality of identified subterranean samples based on the estimated value for the target parameter.

20. The computer-implemented method of claim 19, further comprising:
facilitate identifying an unknown value for the target parameter associated with the unidentified subterranean sample before determining the estimated value for the target parameter.
